(19) United States
(12) Patent Application Publication
(10) Pub. No.: US 2024/0374652 A1
(43) Pub. Date: Nov. 14, 2024

(54) Acellular Placental Therapies

(71) Applicant: Plakous Therapeutics, Inc., Winston-Salem, NC (US)

(72) Inventors: Scott A. Washburn, Pfafftown, NC (US); Seth Tomblyn, Winston-Salem, NC (US)

(21) Appl. No.: 18/690,229

(22) PCT Filed: Sep. 7, 2022
(86) PCT No.: PCT/US2022/042689

§ 371 Date: Mar. 7, 2024

Related U.S. Application Data
(60) Provisional application No. 63/241,453, filed on Sep. 7, 2021.

(51) Int. Cl. A61K 35/50; A61K 45/06; A61P 19/02

(52) U.S. Cl. CPC A61K 35/50; A61K 45/06; A61P 19/02

(57) ABSTRACT

Disclosed herein are methods of treating a condition affecting bone tissue, muscle tissue, or connective tissue using a placenta-derived composition comprising placental tissue and one or more protease inhibitors. Also disclosed herein are methods of treating an individual having a malady comprising arthritis, osteoporosis, or fibrosis with a placenta-derived composition. Further disclosed herein are methods of treating an individual having irradiation damage to bone, muscle, or connective tissue with a placenta-derived composition.

### CROSS-REFERENCE TO RELATED APPLICATION

This application claims the benefit of U.S. Provisional Application No. 63/241,453, filed Sep. 7, 2021, which is incorporated herein by reference in its entirety.

### FIELD

The present disclosure relates generally to biological compositions and methods for their use. More specifically, but not by way of limitation, this disclosure relates to compositions derived from extracts of placental tissue and methods for their use.

### BACKGROUND

The human placenta connects the fetus to the mother's uterine wall and is responsible for the protection and development of the fetus effectively from conception to the time of birth.

Degradation of musculoskeletal tissues, such as bone tissue, muscle tissue, and connective tissue, can result from disease, excessive physical demands, and exposure to radiation. The long term quality of life of a patient can be negatively impacted by degradation of these tissues.

### SUMMARY

Described herein are compositions and associated methods of preparing and using placental tissue products/compositions. Placental tissue may include the placental disc. Placental tissue may include the amniotic sac. The amniotic sac comprises two primary layers, the chorion and the amnion.

The composition described herein may comprise protease inhibitors. In some embodiments, protease inhibitors may be added to placental tissue during the purification process and may not be removed.

In some embodiments, the placental tissue may be processed to isolate desirable proteins and chemokines from the placental tissue. In some embodiments, a method may comprise adding protease inhibitors to a placental product and then administering one or more of gross homogenization and cell lysis. In some embodiments, the method may further comprise separation of fluid from solid cell components, filtration, lyophilization and/or freezing.

In some embodiments, compositions described herein may be used in the treatment of arthritis, wound healing, scar treatment, tissue growth and healing, tissue matrix regeneration and repair, engraftment, inflammation, conditions associated with modulating TGF-β, and/or other protein signaling.

In some embodiments, compositions described herein may be used in the treatment of degenerative conditions that can affect bone tissue, muscle tissue, and fibrous connective tissue (i.e., tendons, ligaments, and cartilage). In some embodiments, compositions described herein may be used in the treatment of osteocytes, chondrocytes, and/or myocytes. In some embodiments, compositions described herein may be used in the treatment of osteoblasts, osteoclasts, and/or fibroblasts. A liquid formulation comprising an acellular extract of placental tissue and a solvent may prevent the onset or reduce the symptoms of a condition of bone, muscle or connective tissue in a subject when a therapeutically effective amount of the liquid formulation is delivered to a subject.

In some embodiments, compositions described herein may be used to aid in repair of musculoskeletal tissue damaged by radiation. Radiation damage of tissue can occur in a subject from total body radiation exposure from a terrorist attack, conventional warhead, or nuclear accident (e.g., aboard a submarine or aircraft carrier or at nuclear/industrial operations). Radiation damage of tissue can occur in patients undergoing cancer treatments. Tissue damage can increase the risk of a subject developing osteoarthritis, osteoporosis, and/or experiencing catastrophic joint failure.

In some embodiments, compositions described herein may be used in the treatment of arthritis. In some embodiments, compositions described herein may be used in the treatment of rheumatoid arthritis, psoriatic arthritis, and/or osteoarthritis. In some embodiments, compositions described herein may be used in the treatment of osteoporosis, osteopenia, non-union fractures, and/or sarcopenia. In some embodiments, compositions described herein may be used in the treatment of a subject undergoing a cartilage transplant, joint reconstruction, or tendon reconstruction. For example, the compositions described herein may be used in the repair and/or reconstruction of a hip, a knee, or an Achilles tendon. In some embodiments, compositions described herein may be used in biological meshes for breast reconstruction, urinary incontinence, and/or hernia repair. In some embodiments, compositions described herein may be used in treatment for skin healing after burns or other skin damaging injuries (e.g., pressure sores, diabetic foot ulcers, surgical incisions, radiation, thermal and chemical burns). In some embodiments, compositions described herein may be used in treatment for fibrosis.

In some embodiments, compositions described herein may be used in the treatment of degenerative conditions that can affect smooth muscle tissue (e.g., lung, intestine, uterus, stomach, and bladder). In some embodiments, compositions described herein may be used in the treatment of degenerative conditions that can affect myocardium tissue. A liquid formulation comprising an acellular extract of placental tissue and a solvent may prevent the onset or reduce the symptoms of a condition of smooth muscle or myocardium tissue in a subject when a therapeutically effective amount of the liquid formulation is delivered to a subject.

In some embodiments, a liquid formulation comprising an acellular extract of placental tissue may be administered orally or systematically to a subject. In some embodiments, a liquid formulation comprising an acellular extract of placental tissue may be administered by local treatment, either topical or by injection, to a subject.

The composition and methods of the present invention may be desirable because of elevated concentrations of proteins relative to conventional compositions that do not use protease inhibitors.

### BRIEF DESCRIPTION OF THE DRAWINGS

FIG. 1 shows the protein concentration amounts of interleukin 2 and macrophage inflammatory protein 3 from placental tissue in both the presence and absence of protease inhibitors.

FIG. 2 shows the protein concentration amounts of granulocyte chemotactic protein 2, monocyte chemotactic protein 1, and macrophage inflammatory protein 1 from placental tissue in both the presence and absence of protease inhibitors.

FIG. 3 shows the protein concentration amounts of interleukin 8 from placental tissue in both the presence and absence of protease inhibitors.

FIG. 4 shows the protein concentration amounts of macrophage inhibitory factor from placental tissue in both the presence and absence of protease inhibitors.

FIGS. 5A-5C shows the results of a wound healing assay using human placental homogenate (HPH) in the presence of a negative control with no serum (5A), in the presence of serum that has been purified in the presence of protease inhibitors (5B), and in the presence of serum that has been purified in the absence of protease inhibitors (5C). As used herein, the terms purified and isolated are interchangeable.

FIG. 6 shows the protein concentration amounts in placental preparation, term amniotic fluid, and 16-20 week amniotic fluid.

FIG. 7 shows variation of specific protein levels for individual, 3-placenta, 4-placenta, 5-placenta and 10-placenta lots.

FIG. 8 shows concentrations of osteoarthritis related proteins within placental compositions with protease inhibitors.

FIG. 9 is an image of radiographic osteoarthritis from the knee of a non-human primate (NHP).

FIG. 10 shows images showing effects of radiation in hip cartilage from non-human primates (NHPs).

FIG. 11 shows the concentration of non-human primate (NHP) chondrocytes when treated with inflammatory solutions, the placental preparation, and inflammatory solutions with the placental preparation in combination compared to a control.

FIG. 12 shows dose dependent alkaline phosphatase (ALP) activity at Day 2 and Day 40 after radiation therapy (RT) in pig chondrocytes.

FIG. 13 shows change in type X collagen mRNA expression at Day 40 after irradiation in pig chondrocytes.

FIG. 14 shows fluorescent images showing nuclear localization of β catenin in pig chondrocytes at Day 1 after 10 Gray (Gy) of γ-rays.

FIG. 15 shows mRNA expression of RUNX 2 at Day 1 after 10 Gy γ-rays.

FIG. 16 shows mRNA expression of LEF-1 at Day 1 after 10 Gy γ-rays.

FIG. 17 shows images of osteoarthritis development in a rat model of traumatic injury.

FIG. 18 shows the OARSI score for a rat model of traumatic injury.

FIG. 19 shows effect of the placental preparation on IL-1 in MMP-13 and MMP-1 from isolated pig chondrocytes with Densitometric Fold Change when exposed to 10 ng/ml of the placental preparation.

FIG. 20 shows the release of GAGs from IL-1-treated pig explants with Densitometric Fold Change when exposed to 10 ng/ml of the placental preparation.

FIG. 21A shows T2-weighted MR images of (A) a rat knee in a sagittal section.

FIG. 21B shows a region of articular cartilage lining the tibial plateau.

FIG. 22, shows T2-relaxation times generated from the region of FIG. 21B.

FIG. 23 shows osteoclast activity of a cultured bone proxy model.

### DETAILED DESCRIPTION

The present disclosure relates to compositions and associated methods of preparing placental tissue products/compositions. The human placenta connects the fetus to the mother's uterine wall and is responsible for the protection and development of the fetus effectively days after conception to the time of birth. During pregnancy, the placenta helps the fetus develop by providing nutrients and oxygen, by performing some immunity functions, and by releasing growth factors and cytokines, small proteins that act to direct cell migration.

During pregnancy, the placenta provides nutrients, growth factors, and cytokines to the fetus via the umbilical cord and amniotic fluid. Both the fetus and placenta express antigens that are disparate from the mother, yet avoid being rejected by the maternal immune system during the pregnancy. The residual cells, transport properties, and limited immune response of placental tissue help make it desirable and/or advantageous to utilize placental tissue in medical applications.

Placental tissue may be typically collected after an elective Cesarean surgery. Placental tissue may also be collected after a normal vaginal delivery. The tissue may be used unmodified in some applications. However, it would be advantageous to improve the performance of placental tissue for enhanced medical applications. For example, it is advantageous and/or desirable to isolate placental tissue proteins, growth factors, and chemokines for various medical applications.

For purposes of describing certain embodiments of the present disclosure, reference is made herein to human placenta tissue. Embodiments of the present disclosure, however, are not limited to comprising naturally occurring human placenta tissue. Embodiments of the present disclosure may include, but are not limited to: natural or synthetic human placenta tissue: natural or synthetic mammalian placenta tissue: natural or synthetic placenta tissue from other animals, e.g., bovine, equine, porcine, ovis, capra, or camelid; and/or natural and/or synthetic compositions having similar properties to placenta tissue.

Placenta tissue comprises the placental disc, the amniotic sac, and umbilical cord, its vessels and Wharton's Jelly cushioning the umbilical cord vessels. The amniotic sac comprises the outer chorion and the inner amnion. The chorion comprises a reticular layer, basement layer, and trophoblast layer. The trophoblast layer may be adhered to the maternal decidua. The umbilical cord connects the placenta to the fetus, and may transport oxygenated blood and nutrients to the fetus. Much of the placental disc is comprised of chorionic villi, which are extensions of the chorionic villous tree. Through these structures, fetal nutrition and exchange of fetal and maternal cytokines and growth factors may occur.

Placenta villi are composed of three layers of components with different cell types in each: (1) syncytiotrophoblasts/cytotrophoblasts that may cover the entire surface of the villous tree and bathe in maternal blood within the intervillous space: (2) mesenchymal cells, mesenchymal derived macrophages (Hofbauer cells), and fibroblasts that may be located within villous core stroma between trophoblasts and fetal vessels; and (3) fetal vascular cells that include vascular smooth muscle cells, perivascular cells (pericytes), and endothelial cells. Hofbauer cells may synthesize VEGF and other proangiogenic factors that initiate vasculogenesis in the placenta.

Both the fetus and placenta may express antigens that are disparate from the mother, yet avoid being rejected by the maternal immune system during the pregnancy. The limited immuno response of the placental tissue is believed to assist the placenta in avoiding fetal rejection during pregnancy.

The transport properties, residual cells, and limited immune response of placental tissue help make it desirable to utilize placental tissue in medical applications. Placental tissue may be collected after an elective Cesarean surgery. Placental tissue may also be collected after a normal vaginal delivery. Placental tissue may be obtained through FDA registered tissue banks. The tissue may be used unmodified in some applications. However, it would be advantageous to improve the performance of the placental tissue for enhanced medical applications.

The placental products/compositions described herein may have beneficial properties relative to those placental preparations of the prior art. For example, the placental products/compositions of the present disclosure include protease inhibitors that are added to placental tissue during the purification process and are not removed. The methods of the disclosure include the treatment of arthritis, wound healing, scar treatment, tissue growth and healing, engraftment, inflammation, and conditions associated with modulating TGF-β and other protein signaling.

To date, various preparations have been purified from the placenta that have been used for a variety of purposes such as treating inflammation, scar treatment, modulating TGF-β signaling, treating apoptosis and associated conditions. However, because governmental regulatory agencies may require more stringent testing conditions and more extensive clinical trials when foreign additives are added to preparations (such as additives to various amniotic or placental preparations), those of skill in the art have been reluctant to include these additives to purified preparations due to the added cost, added regulatory requirements, as well as other factors. However, the preparations created by others that have failed to include and/or contemplate including these additives have also unfortunately failed to unlock the true potential value/uses of these preparations.

### Placental Compositions

In embodiments, the present disclosure relates to placental tissue products and compositions. Placental tissue as used herein comprises chorion frondosum tissue, decidua basalis tissue, and/or interconnecting tissue, which may be in whole or in part. In some embodiments, compositions described herein have beneficial properties relative to conventional placental preparations of the prior art. For example, in some embodiments, the placental products/compositions described herein comprise protease inhibitors that are added to placental tissue during the purification process. In some embodiments, the protease inhibitors may remain in the placental product.

In an embodiment, the protease inhibitors may be removed prior to using the placental product to treat an individual that suffers from various conditions, including arthritis, wound healing, scar treatment, tissue growth and healing, engraftment, inflammation, and conditions associated with modulating TGF-β and other protein signaling. For example, by employing a column that has antibodies that specifically bind the protease inhibitors, the protease inhibitors may be removed prior to treatment of said individual. As used herein, the terms maladies, diseases, and conditions may be used interchangeably.

In an embodiment, the present disclosure relates to purified compositions and placenta/villous chorion preparations i.e., compositions that may be prepared from placenta/villous chorion materials. In some embodiments, at least one component of the purified compositions may be obtained from placenta/villous chorion preparations. In some embodiments, the present disclosure also relates to purified compositions in which at least one component of the purified composition may be obtained from human placenta and chorion. In an embodiment, the present disclosure relates to methods for preparing any of the purified compositions and preparations described herein. The present disclosure also relates to methods for storing and preserving any of the aforementioned purified compositions and preparations. Further, the present disclosure relates to methods for using the aforementioned purified compositions and preparations, including preservative methods, cell culture methods, tissue culture methods, therapeutic methods, prophylactic methods and cosmetic methods.

In an embodiment, methods for reducing or preventing inflammation in a subject comprise providing an effective amount of an inflammation inhibition composition to a subject in need of inflammation inhibition or prevention, where the composition comprises at least one human amniotic material selected from a human amniotic membrane, a human amniotic jelly, a human amniotic stroma, placenta/villous chorion or a combination thereof extracted from a placenta. In some embodiments, the material may be extracted from the human amniotic material. In some embodiments, the composition may comprise, for example, cross-linked high molecular weight hyaluronan (HA), Tumor necrosis factor-stimulated gene 6 (TSG-6), Pentraxin (PTX-3), and Thrombospondin (TSP-1).

The term “effective amount” as used herein, refers to a sufficient amount of an agent, a compound, or ingredients being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. An “effective amount” of a compound/ingredients disclosed herein, is an amount effective to achieve a desired pharmacologic effect or therapeutic improvement without undue adverse side effects. It is understood that “an effect amount” can vary from subject to subject, due to variation in metabolism of the composition, age, weight, general condition of the subject, the condition being treated, the severity of the condition being treated, and the judgment of the prescribing physician.

In some embodiments, the extraction method may comprise, obtaining a human placenta, isolating the human amniotic material from the placenta, and homogenizing the human amniotic material in a suitable buffer, In some embodiments, a frozen or previously-frozen human placenta may be used. If the placenta is frozen, the procedure may comprise thawing the placenta prior to isolating the human amniotic material from the thawed placenta. Optionally the method may further comprise lyophilizing the homogenate to a powder. Optionally, the method may comprise admixing the homogenate or the powder with a pharmaceutically acceptable carrier for a non-solid dosage form or an extended release solid dosage form. In an embodiment, the preparation procedure may substitute the step of lyophilizing the homogenate with the step of centrifuging the homogenate, isolating the supernatant from the centrifuged homogenate, and optionally lyophilizing the supernatant to a powder. In an embodiment, one may alternatively or additionally freeze the centrifuged homogenate. In some embodiments, the composition may be provided as a non-solid dosage form or an extended release solid dosage form.

In an embodiment, the present disclosure relates to adding protease inhibitors to a placental preparation prior to undergoing any of purification steps described herein of the placental preparation. In an embodiment, the protease inhibitors may remain in the placental preparation throughout the entire purification protocol. In some embodiments, the protease inhibitors may remain in the placental preparation that has been purified when the purified product is used for one of the disclosed methods herein. In an embodiment, the protease inhibitors may be added to live cells and/or live tissues when undergoing the purification protocol.

In an embodiment, the present disclosure relates to a cell free composition that comprises one or more of growth factors and/or cytokines derived from placental origin, where said composition comprises at least one protease inhibitor.

In an embodiment, the protease inhibitor may be one of more inhibitors that inhibit serine proteases, cysteine proteases, metalloproteases, aspartic proteases, threonine proteases, or trypsin inhibitors. In some embodiments, the one or more protease inhibitors may inhibit all of serine proteases, cysteine proteases, metalloproteases, aspartic proteases, threonine proteases, and trypsin inhibitors. In some embodiments, the protease inhibitors may inhibit serine proteases and cysteine proteases.

In an embodiment, protease inhibitors that may be used include one or more 4-(2-Aminoethyl) benzenesulfonyl fluoride hydrochloride (AEBSF), Bovine Lung Aprotinin, N-(trans-Epoxysuccinyl)-L-leucine 4-guanidinobutylamide, and Leupeptin. In an embodiment, other protease inhibitors that may be used include N-ethylmaleimide (NEM), phenylmethylsulfonylfluoride (PMSF), ethylenediamine tetraacetic acid (EDTA), ethylene glycol-bis-(2-aminoethyl (ether) NNN′N′-tetraacetic acid, ammonium chloride, boceprevir, danoprevir, narlaprevir, telaprevir, or vaniprevir.

In an embodiment, the placental composition may be derived from tissues and/or cells from any one or more of the chorion, the placenta, the amniotic sheet, the amnion, the umbilical cord, the mesoderm, the yolk sac, the exocoelem, Hofbauer cells, endothelial cells, and/or the endoderm. In one embodiment, the purified placental composition derives from cells and/or tissue from the chorion that has had protease inhibitors added to it prior to any purification steps. In one embodiment, the protease inhibitors that may be added include 4-(2-Aminoethyl) benzenesulfonyl fluoride hydrochloride (AEBSF), Bovine Lung Aprotinin, N-(trans-poxysuccinyl)-L-leucine 4-guanidinobutylamide, and Leupeptin. It has been found that when the chorion is used as the starting material in the purification process, that a more uniform purification product may be attained.

### Method of Purifying Compositions

In an embodiment, the present disclosure relates to a cell free preparation that may preserve the proteins both from the extracellular matrix and intracellular compartments of the placenta. In one embodiment, the present disclosure relates to a cell free preparation that may preserve the proteins found in the trophoblastic cells. In an embodiment, the disclosure relates to a cell free preparation that may preserve the proteins in the cytotrophoblasts, which may synthesize the various cytokines and growth factors for fetal development. Alternatively and/or additionally, the present disclosure relates to a cell free preparation that may preserve the proteins found in the syncytiotrophoblasts, which are pseudocells that may store products of cytotrophoblasts. The syncytiotrophoblast is a multinuclear layer that may form and expand throughout pregnancy by intercellular fusion of the underlying feeder layer of mononuclear villous cytotrophoblasts.

In one embodiment, by adding protease inhibitors throughout the purification process and keeping them as part of the composition, one may be able to isolate compositions having significantly elevated concentrations of proteins relative to those preparations where protease inhibitors are not used. For example, in an embodiment, at least one of the following proteins can be found at elevated levels relative to those purification methods where protease inhibitors are not used: granulocyte chemotactic protein 2 (GCP-2), interleukin 2 (IL-2), interleukin 8 (IL-8), monocyte chemotactic protein 1 (MCP-1), macrophage inhibitory factor (MIF), macrophage inflammatory protein 1 (MEP-1α), and macrophage inflammatory protein 3 (MIP-3a). In some embodiments, several of these proteins are found at elevated concentrations. In some embodiments, all of these proteins are found at elevated concentrations relative to those preparations/compositions that are performed in the absence of protease inhibitors.

In some embodiments, the purification protein concentration may match the cytokines and growth factors that are present in the chorion at the 16th week of human gestation. In an embodiment, the isolated composition may show superior properties compared to conventional preparations/compositions that do not use protease inhibitors. For example, wound healing in the preparations/compositions using the methods of the present disclosure may be unexpectedly superior to compositions isolated without using protease inhibitors.

In some embodiments, the method of purifying the composition may comprise a number of purification steps. In some embodiments, the method of purification may comprise homogenization and cell lysis. In some embodiments, the methods of purification may further comprise tissue isolation. In some embodiments, the placenta may be pretreated to isolate the tissue by removing excess amounts of blood from the tissue. For example, the placenta, which may be stored in a normal saline solution awaiting processing may be placed in a container with a buffered solution, such as phosphate buffered saline solution (PBS), and mixed to remove excess blood. In some embodiments, the container of tissue in solution may be sonicated, placed on a shaking platform, or agitated. In some embodiments, placenta pieces may be grossly homogenized using a laboratory blender or similar means. Optionally, the pieces may be homogenized with 1×PBS comprising protease inhibitor (PI). In some embodiments, the PI may be at a 1× final concentration. Not intending to be bound by theory, the protease inhibitor may prevent the breakdown of proteins, specifically growth factors, chemokines, and cytokines present in the cells of the placenta.

In some embodiments, the homogenized solution may be separated to remove excess blood liberated during homogenization process. In some embodiments, the separation may be performed by filtration or centrifugation, where the fluid may be discarded and the solids retained for further processing. The solids may compact during centrifugation, forming a mass at the bottom of the container. In some embodiments, the solids may be washed or more times to further remove excess blood from the homogenized solids. Optionally, the washed solids may be cooled prior to further processing. In some embodiments, the washed solids may be cooled to less than 10° C.

In some embodiments, cell lysis may be performed by various methods, including but not limited to, high pressure homogenization, freeze/thaw, chemical, sonication, osmotic pressure, high shear mixing, or combinations thereof. Not intending to be bound by theory, cell lysis may release chemokines, cytokines, and growth factors from the cells. In some embodiments, the lysed cells or cellular debris may be separated from fluid. The fluid or supernatant may comprise chemokines, cytokines, and growth factors and in some embodiments, may be retained for further processing.

In some embodiments, the method of purification may further comprise tissue dissection prior to homogenization. The optional dissection may increase the efficiency of the homogenization process. The dissection may also allow for increased methods to be used for homogenization, without a size on tissue feed. As one example, the tissue may be cut into smaller pieces, such as 2 inches by 2 inches. As another example, the tissue may be cut into smaller pieces, such as 1.5 inches by 2 inches or 1 inch by 2 inches or 1 inch by 2.5 inches, or 1.5 inches by 2.5 inches.

In some embodiments, the method of purification may further comprise separation of fluid from solid cell components, for example by filtration or centrifugation. In some embodiments, the method of purification may further comprise lyophilization and/or freezing or some other purification protocol. In some embodiments, the human placental homogenate (HPH) preparations may be preserved by lyophilization placing in sterile vials, placing in sterile vials in liquid form for later use, spray drying, or other methods known by one skilled in the art. In some embodiments, the HPH may be added to other placental products.

In some embodiments, the purification steps may comprise: (1) tissue isolation: (2) homogenization: (3) cell lysis: (4) separation of fluid from solid cell components; and (5) lyophilization and/or freezing or some other purification protocol.

In some embodiments, the protease inhibitors may be added after the tissue has been isolated. In some embodiments, the protease inhibitors may be present during cell lysis. In an embodiment, the protease inhibitors may be present throughout the full purification of the composition, including when the composition may be used in the methods described herein.

### Compositions for Pharmaceutical Use

In an embodiment, the present disclosure relates to generating pharmaceutical compositions. In some embodiments, the pharmaceutical composition may contain pharmaceutically acceptable salts, solvates, and products thereof, and may contain diluents, excipients, carriers, or other substances necessary to increase the bioavailability or extend the lifetime of the ingredients/compounds of the present disclosure.

In some embodiments, subjects that may be treated by the ingredients/compounds and compositions of the present disclosure include, but are not limited to, horses, cows, sheep, pigs, mice, dogs, cats, primates such as chimpanzees, gorillas, rhesus monkeys, and humans. In an embodiment, a subject may be a human in need of cancer treatment.

The pharmaceutical compositions containing the ingredients/compounds of the disclosure may be in a form suitable for injection either by itself or alternatively, using liposomes, micelles, and/or nanospheres. Alternatively, in some embodiments, the pharmaceutical compositions may be in a form that allows it to be administered topically.

Alternatively, in some embodiments, compositions intended for injection may be prepared according to any known method, and such compositions may comprise one or more agents selected from the group consisting of solvents, co-solvents, solubilizing agents, wetting agents, suspending agents, emulsifying agents, thickening agents, chelating agents, antioxidants, reducing agents, antimicrobial preservatives, buffers, pH adjusting agents, bulking agents, protectants, tonicity adjustors, and special additives. Moreover, other non-toxic pharmaceutically-acceptable excipients which are suitable for the manufacture of injectables may be used.

In some embodiments, the composition may be aqueous suspensions comprising the active placental ingredients in an admixture with excipients suitable for the manufacture of aqueous suspensions. Such excipients may comprise suspending agents and/or dispersing/wetting agents. Suspending agents may include, for example, sodium carboxy methylcellulose, methylcellulose, hydroxypropylmethylcellulose, sodium alginate, polyvinylpyrrolidone, gum tragacanth and gum acacia. Dispersing or wetting agents may be a naturally-occurring phosphatide, such as lecithin, or a synthesized condensation product, such as condensation products of an alkylene oxide with fatty acids, for example, polyoxyethylene stearate, condensation products of ethylene oxide with long chain aliphatic alcohols, for example, heptadecaethyl eneoxycethanol, condensation products of ethylene oxide with partial esters derived from fatty acids and a hexitol, for example, polyoxyethylene sorbitol monooleate, or condensation products of ethylene oxide with partial esters derived from fatty acids and hexitol anhydrides, for example, polyethylene sorbitan monooleate. In some embodiments, the aqueous suspensions may also contain one or more coloring agents.

In some embodiments, oily suspensions may be formulated by suspending the active ingredient in a vegetable oil, for example, arachis oil, olive oil, sesame oil or coconut oil, or in a mineral oil such as a liquid paraffin. The oily suspensions may comprise a thickening agent, for example, beeswax, hard paraffin or cetyl alcohol. In some embodiments, sweetening agents and flavoring agents may be added to provide a palatable oral preparation. In some embodiments, these compositions may be preserved by the addition of an anti-oxidant such as ascorbic acid.

In some embodiments, dispersible powders and granules suitable for preparation of an aqueous suspension by the addition of water may provide the active ingredients in admixture with a dispersing or wetting agent, suspending agent and one or more preservatives. Suitable dispersing or wetting agents and suspending agents are exemplified by those already mentioned above. In some embodiments, additional excipients, for example, sweetening, flavoring, and coloring agents may also be present.

In some embodiments, the pharmaceutical compositions of the disclosure may also be in the form of oil-in-water emulsions. The oily phase may comprise a vegetable oil, for example, olive oil or arachis oil, a mineral oil, for example, a liquid paraffin, or a mixture thereof. Optionally, suitable emulsifying agents may be naturally-occurring gums, for example, gum acacia or gum tragacanth, naturally-occurring phosphatides, for example, soy bean, lecithin, and esters, partial esters derived from fatty acids and hexitol anhydrides, for example, sorbitan monooleate, or condensation products of said partial esters with ethylene oxide, for example, polyoxyethylene sorbitan monooleate. In some embodiments, the pharmaceutical compositions may be in the form of a sterile injectable aqueous or oleaginous suspension. This suspension may be formulated according to the known methods using suitable dispersing or wetting agents and suspending agents described above. Optionally, the sterile injectable preparation may also be a sterile injectable solution or suspension in a non-toxic parenterally-acceptable diluent or solvent, for example, as a solution in 1,3-butanediol. In some embodiments, among the acceptable vehicles and solvents that may be employed are water, sterile water for injection (SWFI), Ringer's solution, and isotonic sodium chloride solution. In addition, sterile, in some embodiments, fixed oils may be conveniently employed as solvent or suspending medium. For this purpose, any bland fixed oil may be employed using synthetic mono- or diglycerides. In addition, in some embodiments, fatty acids such as oleic acid may be used in the preparation of injectables.

In some embodiments, a solution of the disclosure may be provided in a sealed container, especially one made of glass, either in a unit dosage form or in a multiple dosage form.

In some embodiments, any pharmaceutically acceptable salt of the ingredients of the present disclosure may be used for preparing a solution of the disclosure. Examples of suitable salts may be, for instance, the salts with mineral inorganic acids such as hydrochloric, hydrobromic, sulfuric, phosphoric, nitric and the like, and the salts with certain organic acids such as acetic, succinic, tartaric, ascorbic, citric, glutamic, benzoic, methanesulfonic, ethanesulfonic and the like. In an embodiment, the ingredients of the disclosure may be a hydrochloric acid salt including a mono, di, or trihydrochloride.

In some embodiments, the composition may also comprise one or more additional components such as a co-solubilizing agent, which may be the same as a solvent, a tonicity adjustment agent, a stabilizing agent, a preservative, or mixtures thereof. Examples of solvents, co-solubilizing agents, tonicity adjustment agents, stabilizing agents and preservatives which may suitable for a solution formulation are described below.

In some embodiments, suitable solvents and co-solubilizing agents may include, but are not limited to, water: sterile water for injection (SWFI): physiological saline: alcohols, e.g., ethanol, benzyl alcohol and the like: glycols and polyalcohols, e.g., propyleneglycol, glycerin and the like: esters of polyalcohols, e.g., diacetine, triacetine and the like; polyglycols and polyethers, e.g., polyethyleneglycol 400, propyleneglycol methylethers and the like: dioxolanes, e.g., isopropylidenglycerin and the like: dimethylisosorbide: pyrrolidone derivatives, e.g., 2-pyrrolidone, N-methyl-2-pyrrolidone, polyvinylpyrrolidone (co-solubilizing agent only) and the like: polyoxyethylenated fatty alcohols: esters of polyoxyethylenated fatty acids: polysorbates, e.g., Tween™, polyoxyethylene derivatives of polypropyleneglycols, e.g., Pluronics™. Suitable tonicity adjustment agents may include, but are not limited to, pharmaceutically acceptable inorganic chlorides, e.g., sodium chloride; dextrose: lactose: mannitol: sorbitol and the like.

In some embodiments, preservatives suitable for physiological administration may be, for example, esters of parahydroxy benzoic acid, e.g., methyl, ethyl, propyl and butyl esters, or mixtures of them, chlorocresol and the like.

In some embodiments, suitable stabilizing agents include, but are not limited to, monosaccharides, e.g., galactose, fructose, and fucose, disaccharides, e.g., lactose, polysaccharides, e.g., dextran, cyclic oligosaccharides, e.g., alpha-, beta-, gamma-cyclodextrin, aliphatic polyols, e.g., mannitol, sorbitol, and thioglycerol, cyclic polyols, e.g., inositol, and organic solvents, e.g., ethyl alcohol and glycerol.

In some embodiments, the above-mentioned solvents and co-solubilizing agents, tonicity adjustment agents, stabilizing agents and preservatives can be used alone or as a mixture of two or more of them in a solution formulation.

In an embodiment, a pharmaceutical solution formulation may comprise the compositions described herein or pharmaceutically acceptable salts thereof, and an agent selected from the group consisting of sodium chloride solution (i.e., physiological saline), dextrose, mannitol, or sorbitol, wherein the agent is in an amount of less than or equal to 5%. In some embodiments, the pH of such a formulation may also be adjusted to improve the storage stability using a pharmaceutically acceptable acid or base.

In some embodiments, the concentration of active placental ingredients in the formulation or a pharmaceutically acceptable salt thereof may be less than 100 mg/mL. For example, the concentration may be less than 100 mg/mL, may be less than 75 mg/mL, less than 50 mg/mL, less than 10 mg/mL, or less than 10 mg/mL and greater than 0.01 mg/mL, or between 0.5 mg/mL and 5 mg/mL, or between 1 mg/mL and 3 mg/mL. In an embodiment, the concentration that is used may be the ideal concentration to sufficiently treat the disease and/or condition that is being treated.

In some embodiments, for solution formulations, various compositions of the present disclosure may be more soluble or stable for longer periods in solutions at a pH lower than 6. Further, in some embodiments, acid salts of the ingredients in the compositions of the present disclosure may be more soluble in aqueous solutions than their free base counter parts, but when the acid salts are added to aqueous solutions the pH of the solution may be too low to be suitable for administration. Thus, in some embodiments, solution formulations having a pH above pH 4.5 may be combined prior to administration with a diluent solution of pH greater than 7 such that the pH of the combination formulation administered may be pH 4.5 or higher. In one embodiment, the diluent solution comprises a pharmaceutically acceptable base such as sodium hydroxide. In another embodiment, the diluent solution may be at pH of between 10 and 12. In another embodiment, the pH of the combined formulation administered may be greater than 5.0. In another embodiment, the pH of the combined formulation administered may be between pH 5.0 and 7.0.

In some embodiments, the compositions described herein may be passed through a filter. Optionally, one or more additional components such as co-solubilizing agents, tonicity adjustment agents, stabilizing agents and preservatives, for instance of the kind previously specified, may be added to the solution prior to passing the compositions through a sterilizing filter.

### Methods of Using the Composition

In some embodiments, the compositions described herein may be used in combination therapy with other compositions that are known to be used on the diseases and/or conditions that the present disclosure is designed to address. The dosages of the co-administered compounds/compositions may vary depending on the type of co-drug employed, on the specific drug employed, on the disease or condition being treated and so forth. In addition, in some embodiments, when co-administered with one or more biologically active agents, the compounds/compositions provided therein may be administered either simultaneously with the biologically active agent(s) of the present disclosure or may be administered sequentially. If administered sequentially, the attending physician may determine the appropriate sequence of administering protein in combination with the biologically active agent(s).

In an embodiment, multiple therapeutic agents may be administered in any order or even simultaneously. If administered simultaneously, the multiple therapeutic agents may be provided in a single, unified form, or in multiple forms (by way of example only, either as a single pill or as two separate pills). Optionally, one of the therapeutic agents may be given in multiple doses, or both may be given as multiple doses. If not simultaneous, the timing between the multiple doses may vary from more than zero weeks to less than four weeks. In addition, in some embodiments, the combination methods, compositions and formulations are not to be limited to the use of only two agents: the use of multiple therapeutic combinations may also be envisioned.

In an embodiment, pharmaceutical agents which comprise the combination therapy disclosed herein may be a combined dosage form or in separate dosage forms intended for substantially simultaneous administration. Optionally, the pharmaceutical agents comprising the combination therapy may also be administered sequentially, with either therapeutic compound being administered by a regimen calling for two-step administration. In some embodiments, the two-step administration regimen may call for sequential administration of the active agents or spaced-apart administration of the separate active agents. In some embodiments, the time period between the multiple administration steps may range from, a few minutes to several hours, depending upon the properties of each pharmaceutical agent, such as potency, solubility, bioavailability, plasma half-life and kinetic profile of the pharmaceutical agent.

Moreover, the compositions of the present disclosure and purified compositions described herein also may be used in combination with procedures that may provide additional or synergistic benefit to the patient. By way of example only, patients are expected to find therapeutic and/or prophylactic benefit in the methods described herein, wherein pharmaceutical composition of a compound disclosed herein and/or combinations with other therapeutics may be combined with genetic testing to determine whether that individual is a carrier of a mutant gene that is known to be correlated with certain diseases or conditions.

The compositions of the present disclosure described herein and combination therapies may be administered before, during or after the occurrence of a disease or condition, and the timing of administering the composition containing one or more active ingredients may vary. Thus, for example, the compounds may be used as a prophylactic and may be administered continuously to subjects with a propensity to develop conditions or diseases in order to prevent the occurrence of the disease or condition. In some embodiments, the formulations and compositions of the present disclosure can be administered to a subject during or as soon as possible after the onset of the symptoms. For example, the administration of the formulations and compositions may be initiated within the first 48 hours of the onset of the symptoms, or within the first 48 hours of the onset of the symptoms, or within the first 6 hours of the onset of the symptoms, or within 3 hours of the onset of the symptoms. In some embodiments, the initial administration may be via any route practical, such as, for example, an intravenous injection, a bolus injection, infusion over 5 minutes to about 5 hours, a pill, a capsule, transdermal patch, buccal delivery, topical administration and the like, or any combination thereof. In some embodiments, the formulations and compositions may be administered as soon as is practicable after the onset of a disease or condition is detected or suspected, and for a length of time necessary for the treatment of the disease, such as, for example, from about 1 month to about 3 months. The length of treatment can vary for each subject, and the length may be determined using the known criteria. For example, the compositions containing ingredients that treat the disease or condition may be administered for at least 2 weeks, or about 1 month to about 5 years, or from about 1 month to about 3 years.

The present disclosure also relates to using the compositions and formulations described herein in kits that may be used to treat diseases and conditions that require treatment. In some embodiments, such kits can include a carrier, package, or container that may be compartmentalized to receive one or more containers such as vials, tubes, and the like. Optionally, each of the container(s) may include one of the separate elements to be used in a method described herein. Suitable containers may include, for example, bottles, vials, syringes, and test tubes. The containers may be formed from a variety of materials such as glass or plastic.

In some embodiments, the articles of manufacture provided herein may contain packaging materials. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compositions provided herein may be contemplated as are a variety of treatments for any disease, disorder, or condition.

Various methods of use may be contemplated in the present disclosure. Included in the methods are methods of making and using the placental preparations of the instant disclosure including the treatment of individuals that are in need of said treatment.

### Placental Preparation

In an embodiment, the present disclosure relates to a placental preparation wherein said placental preparation may be derived from a placental tissue, where the placental tissue may be subjected to processing steps comprising gross homogenization and cell lysis. In some embodiments, the placental tissue may be subjected to processing steps further comprising separation of fluid from solid cell components, filtration, and lyophilization and/or freeze to generate the placental preparation. In some embodiments, the placental preparation further comprises one or more protease inhibitors. In some embodiments, the placental preparation may be derived from placental tissue, where the placental tissue has undergone the following steps in the order of: (a) homogenization: (b) cell lysis: (c) separation of fluid from solid cell components; and (d) lyophilization and/or freeze to generate the placental preparation, where the placental preparation further comprises one or more protease inhibitors. The placental tissue may undergo one or more freeze-thaw cycles. In some embodiments, the preparation may be filtered after the separation and before the lyophilization steps.

In some embodiments, the placental preparation comprises adding the one or more protease inhibitors prior to gross homogenization. In some embodiments, the one or more protease inhibitors comprise one or more of 4-(2-Aminoethyl) benzenesulfonyl fluoride hydrochloride (AEBSF), Bovine Lung Aprotinin, N-(trans-Epoxysuccinyl)-L-leucine 4-guanidinobutylamide, and Leupeptin. Optionally, the one or more protease inhibitors may be selected from the group consisting of 4-(2-Aminoethyl) benzenesulfonyl fluoride hydrochloride (AEBSF), Bovine Lung Aprotinin, N-(trans-Epoxysuccinyl)-L-leucine 4-guanidinobutylamide, and Leupeptin.

In some embodiments, the placental tissue may be the chorion. In some embodiments, the placental preparation may further comprise diluents, excipients, or carriers. In some embodiments, the placental preparation may comprise higher concentration amounts of interleukins relative to preparations that are prepared in the absence of protease inhibitors. In some embodiments, other proteins present in the preparation comprising protease inhibitors may be isolated in higher concentrations relative to proteins in the preparation that are isolated in the absence of protease inhibitors. In some embodiments, the protease inhibitors may not be removed from the placental preparation.

In an embodiment, the present disclosure relates to a method of treating an individual that may have a malady selected from the group comprising arthritis, wound healing, scar treatment, tissue growth, tissue healing, tissue matrix regeneration/repair, engraftment, inflammation, and conditions associated with modulating TGF-β signaling wherein the method comprises administering the placental preparation as enumerated herein. Tissue is used herein in accordance with its generally accepted meaning in biology, i.e. an ensemble of similar cells. Specific tissues include, but are not limited to, muscle, nerve, bony, connective, epithelial, vascular, and the like.

In an embodiment, the present disclosure relates to a method of treating an individual that may have a malady selected from the group comprising arthritis, wound healing, scar treatment, tissue growth, tissue healing, tissue matrix regeneration/repair, engraftment, inflammation, and conditions associated with modulating TGF-β and other protein signaling wherein said method comprises administering to said individual an effective amount of a placental preparation that may be derived from placental tissue, where the placental tissue may be subjected to processing steps comprising gross homogenization and cell lysis. In some embodiments, the processing steps may further comprise separation of fluid from solid cell components, filtration, and lyophilization and/or freeze to generate the placental preparation. In some embodiments, the placental preparation may further comprise one or more protease inhibitors. In some embodiments of said method, the one or more protease inhibitors may be added prior to gross homogenization. In some embodiments, the one or more protease inhibitors may be selected from the group comprising 4-(2-Aminoethyl) henzenesulfonyl fluoride hydrochloride (AEBSF), Bovine Lung Aprotinin, N-(trans-Epoxysuccinyl)-L-leucine 4-guanidinobutylamide, and Leupeptin. In some embodiments, the placental tissue may be the chorion. In some embodiments, the one or more protease inhibitors may be removed prior to administration of the placental preparation to said individual.

In an embodiment, the present disclosure relates to a method of making a placental preparation for treating an individual that may have a malady selected from the group comprising arthritis, wound healing, scar treatment, tissue growth, tissue healing, tissue matrix regeneration/repair, engraftment, inflammation, and conditions associated with modulating TGF-β signaling, wherein said method may comprise: procuring placental tissue and adding protease inhibitors to the placental tissue and then administering one or more of the following steps: (a) gross homogenization: (b) cell lysis: (c) separation of fluid from solid cell components; and (d) lyophilization and/or freeze to generate the placental preparation. In some embodiments, a filtration step may be performed after the separation and before the lyophilization step.

In some embodiments, the separation step may be performed by centrifugation. In some embodiments, the lyophilization step may be performed at reduced pressure. For example, the lyophilization step may be performed at 0.5 atmospheres or less, or 0.3 atmospheres or less, or 0.2 atmospheres or less, or 0.1 atmospheres or less, or 0.05 atmospheres or less, or 0.01 atmospheres or less, or 0.001 or less atmospheres. In some embodiments, the placental tissue may comprise a chorion. In some embodiments, the placental tissue may consist of the chorion.

In some embodiments, compositions described herein may be used in the treatment of degenerative conditions that can affect bone tissue, muscle tissue, and fibrous connective tissue (i.e., tendons, ligaments, and cartilage). In some embodiments, compositions described herein may be used in the treatment of osteocytes, chondrocytes, and/or myocytes. In some embodiments, compositions described herein may be used in the treatment of osteoblasts, osteoclasts, and/or fibroblasts. A liquid formulation comprising an acellular extract of placental tissue and a solvent may prevent the onset or reduce the symptoms of a condition of bone, muscle or connective tissue in a subject when a therapeutically effective amount of the liquid formulation is delivered to a subject.

In some embodiments, compositions described herein may be used to aid in repair of musculoskeletal tissue damaged by radiation. Radiation damage of tissue can occur in a subject from total body radiation exposure from a terrorist attack, conventional warhead, or nuclear accident. Radiation damage of tissue can occur in patients undergoing cancer treatments. Joint degradation and arthritis can occur after cancer treatment, including total body irradiation (TBI). Survivors are fifty times more likely than healthy individuals to require joint replacement. Tissue damage may increase the risk of a subject developing osteoarthritis, osteoporosis, and/or experiencing catastrophic joint failure. For example, hip osteoarthritis may be a risk following targeted radiation treatment of prostate cancer. Tissue damage may occur from extraordinary physical demands or intensive training and recreation activities in subjects. For example, tissue damage could be an anterior cruciate ligament (ACL) injury. In some examples, ACL injury may initiate the cascade of knee joint damage and arthritis. In some examples, the combination of radiation exposure and knee injury may increase both the incidence and severity of knee joint damage and arthritis in patients.

In some embodiments, compositions described herein may be used in the treatment of joint degradation leading to arthritis. Not intending to be bound by theory, joint degradation leading to arthritis and catastrophic joint failure may be a major source of morbidity among cancer survivors treated with total or partial body irradiation, thus, even therapeutic doses of radiation could represent a substantial risk to the joint health of patients.

In some embodiments, calcification of articular cartilage can be a characteristic of osteoarthritis and can contribute to cartilage degradation. Nanocomputed tomography (nano-CT) has shown microcalcifications in the knee joints of rats 9 months after receiving a survivable 3 Gy TBI dose of X-rays. Clinically, articular cartilage calcification and arthritis have been described in adult knees, hips, and hands after radiation exposure. Knee and hip joint degeneration and the resulting chronic, persistent pain may be common following therapeutic TBI. Articular cartilage calcification and degradation, a late degenerative consequence of radiation, may be increased by an initial hypertrophic response of articular cartilage to radiation. Calcifications may be a characteristic of a hypertrophic phenotype in cartilage. Chondrocyte hypertrophy in articular cartilage may be a pathologic process associated with matrix degradation, MMP-13 production, calcification, and arthritis. In some examples, hypertrophic chondrocytes may exhibit (1) reduced expression and production of GAGs and type II collagen, (2) increased expression and production of MMP-13 and type X collagen, and (3) increased production and activity of alkaline phosphatase (ALP) which facilitates calcification. Radiation induced bone loss can lead to fractures which can reduce the quality of life for patients undergoing radiation treatment for cancer. The Wingless/Integrated (Wnt) signaling pathway has been implicated in cartilage degradation during conditions such as osteoarthritis. Wnt signaling may be related to matrix degradation through increased MMP production, and may induce chondrocyte hypertrophy. Activation of the Wnt pathway may result in increased translocation of β-catenin to the nucleus where it can bind to the transcription factors, lymphoid enhancer factor-1 (LEF-1) and t-cell factor (TCF) and may promote expression of RUNX2 (RUNX Family Transcription Factor 2), which may increase the expression of MMP-13 and collagen X associated with cartilage degradation and chondrocyte hypertrophy.

In some embodiments, compositions described herein may be used in the treatment of arthritis. In some embodiments, compositions described herein may be used in the treatment of rheumatoid arthritis, psoriatic arthritis, and/or osteoarthritis. In some embodiments, compositions described herein may be used in the treatment of osteoporosis, osteopenia, non-union fractures, and/or sarcopenia. In some embodiments, compositions described herein may be used in the treatment of a subject undergoing a cartilage transplant, joint reconstruction, or tendon reconstruction. For example, the compositions described herein may be used in the repair and/or reconstruction of a hip, a knee, or an Achilles tendon. In some embodiments, compositions described herein may be used in biological meshes for breast reconstruction, urinary incontinence, and/or hernia repair. In some embodiments, compositions described herein may be used in treatment for skin healing after burns or other skin damaging injuries (e.g., pressure sores, diabetic foot ulcers, surgical incisions, radiation, thermal and chemical burns). In some embodiments, compositions described herein may be used in treatment for fibrosis.

In some embodiments, proteins present in the methods described herein may interfere with osteoarthritis (OA) mechanism and reduce OA severity. IL-10 and Sirtuin-1 (SIRT1) may have chondroprotective properties. IL-10 may act as an antagonist of TNF-alpha and may inhibit apoptosis of chondrocytes. Activated SIRT1 may protect extracellular matrix and stimulate mesenchymal signaling cell differentiation, among several roles that inhibit the progression of OA. Insulin-like Growth Factor 1 (IGF1) and Insulin-like Growth Factor 2 (IGF2) may stimulate chondrocyte growth. Bone Morphogenic Protein 2 (BMP2) may be expressed in the chondrogenic process and histological scores and promote subchondral bone regeneration. Bone Morphogenic Protein 7 (BMP7) may stimulate extracellular matrix (ECM) production and cell proliferation of chondrocytes. Transforming growth factor (TGF-β) may stimulate collagen production in chondrocytes and proteoglycan synthesis in cartilage. Lower endogenous TGF-β levels may be associated with OA. In some embodiments, compositions described herein may be used in the treatment of degenerative conditions that can affect smooth muscle tissue (e.g., lung, intestine, uterus, stomach, and bladder). In some embodiments, compositions described herein may be used in the treatment of degenerative conditions that can affect myocardium tissue. A liquid formulation comprising an acellular extract of placental tissue and a solvent may prevent the onset or reduce the symptoms of a condition of smooth muscle or myocardium tissue in a subject when a therapeutically effective amount of the liquid formulation is delivered to a subject.

### EXAMPLES

### Example 1: Procurement and Isolation of a Placental Tissue

Placenta/amniotic membrane was procured and the human placental homogenate was isolated as follows. The placenta/amniotic membrane was procured from a contract hospital via a placenta donation. The donor was screened according to American Association of Tissue Banks (AATB) guidelines. The placenta/amniotic membrane was placed in 0.9% normal saline (NS, 154 mM NaCl, 308 mOsm/L). The placenta was removed from the 0.9% NS, the placental disc was separated from the amniotic membrane, chorion, and umbilical cord, and dissected into pieces approximately 1.5 inches by 2 inches. These smaller pieces of placental disc were placed in a container with approximately 400 mL of 1× phosphate buffered saline (PBS, 137 mM NaCl, 2.7 mM KCl, 4.3 mM Na₂HPO₄, 1.47 mM KH₂PO₄, pH 7.4). The container was placed on a shaking platform and shaken at 120 rpm for 12 to 24 hrs. The placental disc pieces were grossly homogenized using a laboratory blender with 1×PBS or 1×PBS that contained protease inhibitor (PI) at a 1× final concentration (AEBSF 500 uM, Aprotinin 150 nM, E-64 1 uM, Leupeptin 1 uM). The gross homogenate was placed into 250 mL containers and centrifuged for 10 minutes at 4,000 times gravity. The gross homogenate was poured into the container for a ratio of approximately 1:5, tissue volume: total volume. The container was vortexed/shaken to re-suspend the pellet. The suspension is centrifuged for 10 minutes at 4,000 times gravity. This wash procedure is repeated for a total of three washes. The mixture was not centrifuged after the last wash. The washes removed blood from the tissue. The contents of the 250 mL containers were diluted with either 1×PBS or IX PBS-1×PI to double the volume. The mixture was then placed into a reservoir feed connected to a high-pressure homogenizer. The homogenate was then placed into 250 ml containers and centrifuged at 15,000 times gravity for 10 minutes.

This procedure was performed on 6 placental discs. At the gross homogenization step, the placental discs were split into two groups, one group that was finished with 1×PBS and another that was finished with 1×PBS-1×PI.

A Bio-Plex MAGPIX Multiplex Reader (Bio-Rad Laboratories, Hercules, CA) was used to quantify the amounts of cytokines and growth factors present in the solution extracted from the placental disc. A Bio-Plex Pro Human Chemokine plane, 40-plex assay (Bio-Rad Laboratories, Hercules, CA) was obtained. The standards were diluted in appropriate diluent. Assay beads were diluted and 50 μL was added to each well of the 96-well plate and washed twice with assay buffer. The plate was placed on a magnetic plate holder and the solution removed from the wells. The standards, samples, and blanks were loaded into each respective well of the 96-well plate and incubated for 1 hour at room temperature (note: duplicate measurements were performed on each standard, sample and blank). The plate was washed by placing the plate on the magnetic plate holder, removing the solution, washing with wash buffer, and removing the wash buffer by placing the well plate on the magnetic plate holder. The detection antibodies were added to each well and incubated for 30 minutes at room temperature. The plate was washed 3 times with wash buffer and the streptavidin-PE indicator was added to each well. The plate was incubated for 10 minutes in the dark at room temperature. The plate was washed 3 times in assay buffer and measurements were taken on the MAGPIX multiplex reader. Data was analyzed on Bio-Plex reader software (Bio-Rad Laboratories, Hercules, CA) and a two-way ANOVA was utilized to compare the treatment groups.

Table 1 below shows data with the concentrations of proteins isolated from placenta in the presence and absence of protease inhibitors.

TABLE 1

| | Concentration in the | Concentration in the |
|---|---|---|
| | Presence of Protease | Absence of Protease |
| Protein | Inhibitors (pg/mL) | Inhibitors (pg/mL) |
| GCP-2 | 432.4 | 299.1 |
| IL-2 | 22.3 | 18.0 |
| IL-8 | 30,851.4 | 2423.7 |
| MCP-1 | 1854.5 | 449.8 |
| MIF | 478,523.1 | 336,076.8 |
| MIP-lα | 293.6 | 193.4 |
| MIP-3α | 58.2 | 27.6 |

The data in Table 1 shows that the addition of protease inhibitors increases the amount of the various proteins isolated from the placenta. Not intending to be bound by theory, the increased concentration of proteins may increase the bioavailability of various proteins to aid in treating diseases and/or conditions.

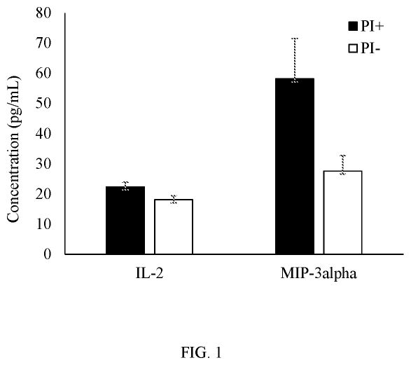

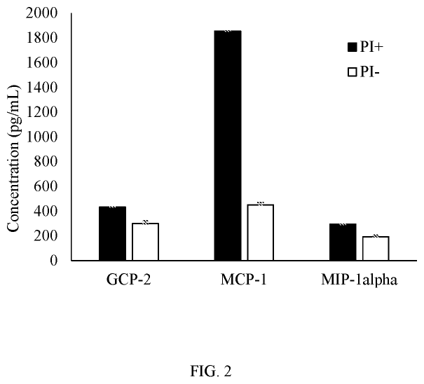

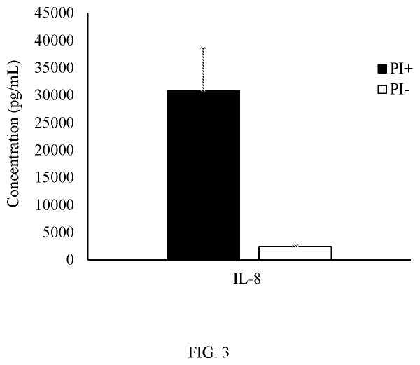

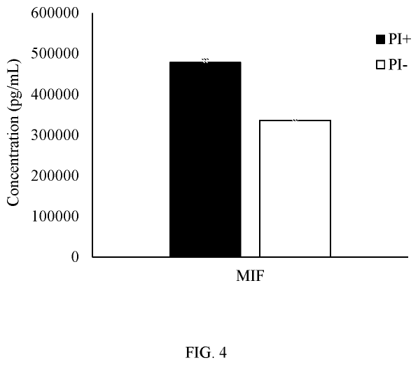

FIGS. 1-4 show the results of assays determining the amount of various amniotic proteins in the presence of protease inhibitors (dark bar) and absence of protease inhibitors (light bar) of protease inhibitors. FIG. 1 shows the isolated protein concentration amounts of interleukin 2 (IL-2) and macrophage inflammatory protein 3 (MIP-3a) from placental tissue. FIG. 2 shows the isolated protein concentration amounts of granulocyte chemotactic protein 2 (GCP-2), monocyte chemotactic protein 1 (MCP-1), and macrophage inflammatory protein 1 (MIP-1α) from placental tissue. FIG. 3 shows the isolated protein concentration amounts of interleukin 8 (IL-8) from placental tissue. FIG. 4 shows the isolated protein concentration amounts of macrophage inhibitory factor (MIF) from placental tissue. The compositions comprising protease inhibitors all showed a statistically significant increase in the isolated protein concentration as compared to those without protease inhibitors. The smallest increase was with IL-2, which produced a nearly 24% increase in IL-2 concentration in the composition comprising protease inhibitors, with a p-value less than 0.05 (FIG. 1). A similar increase in protein concentration was shown for MIF, GCP-2, and MIP-1α, with each showing increases of 42-52% in compositions comprising protease inhibitors over those without protease inhibitors. The p-value for GCP-2 and MIP-1α was less than 0.01: the p-value for MIF was less than 0.05 (FIG. 2 and FIG. 4). A significant increase in protein concentration was shown for MIP-3α and MCP-1 in the protease inhibited compositions with each more than doubling the protein concentration, with increases of 111% and 312%, respectively. The p-value for MIP-3a was less than 0.05: the p-value for MCP-1 was less than 0.01 (FIG. 1 and FIG. 2). A striking increase in protein concentration was shown for IL-8, with an increase of 1173% in the composition comprising protease inhibitors over that without protease inhibitors, with a p-value of less than 0.05 (FIG. 3).

### Example 2: Illustrative Method of One Process of the Disclosure Useful in Making the Placental Preparations Described Herein

The placenta/amniotic membrane is procured from a contract hospital by a placenta donation coordinator. The coordinator/QA department screens the donor according to AATB and company standards. The coordinator places the placenta/amniotic membrane in 0.9% normal saline (NS).

Placenta is removed from the 0.9% NS and dissected into approximately 1.5 inch by 2 inch pieces. Smaller pieces are placed in container with approximately 400 mL of 1×phosphate buffered saline (PBS). The container is placed on a shaking platform shaken at 120 rpm for 12-24 hrs. Shaking the tissue in PBS removes gross amounts of blood from the tissue.

Gross homogenization: Placenta pieces are grossly homogenized using a laboratory blender with 1×PBS that also contains protease inhibitor (P1) at a 1× final concentration. The protease inhibitor may be utilized to prevent the breakdown of proteins, specifically growth factors, chemokines, and cytokines present in the cells of the placenta.

Centrifugation: The gross homogenate is placed into 250 mL containers and centrifuged for 5 minutes at 10000×g. This step separates the bloody fluid from the blended tissue. The blended tissue may form a pellet at the bottom of the container. The bloody fluid is discarded.

Wash: 1×PBS with 1×PI is poured into the container for a ratio of ˜1:5, tissue volume: total volume. The container is vortexed/shaken to re-suspend the pellet. The suspension is centrifuged for 5 minutes at 4000×g. This wash procedure is repeated for a total of three washes. The mixture is not centrifuged after the last wash. These washes remove blood from the tissue. The mixture is cooled to 4° C.

Cell lysis is accomplished by one of the following procedures. Cell lysis may release chemokines, cytokines, and growth factors from the cells.

High Pressure Homogenization: The contents of the 250 mL containers are diluted with 1×PBS with 1×PI to double the volume. The mixture is then placed into cooled reservoir feed connected to a high-pressure homogenizer. The purpose of this step is to lyse any unlysed cells. The pressure and number of passes will be optimized by tests for chemokine and cytokine levels as well as bioactivity.

Freeze/Thaw: The contents of the 250 mL containers are poured into sterile bags and double sealed using a heat sealer. After sealing, the bags are placed on a tray in a −30° C. freezer. The number of bags varies based on total volume of gross homogenate mixture. The gross homogenate is poured into the bags to a volume that results in approximately 1-inch thick bags when laid on the tray. The bags of frozen gross homogenate are removed from the freezer and placed in a warm water bath 37.8° C. to 43.3° C. The homogenate is removed from the warm water bath as soon as it is thawed completely. The thawed mixture is poured into a container and blended using a handheld homogenizer on high speed. The blended mixture is placed into a sterile bag and sealed. This procedure is repeated for a total of up to 3 freeze/thaws.

Chemical Lysis: The gross homogenate is centrifuged and the supernatant is discarded. The pellet is washed with Bio-Rad cell wash buffer. The volume of cell wash buffer varies based on tissue volume. Two times the tissue volume of buffer is added. The pellet and buffer are vortexed/shaken to re-suspend the pellet. The mixture is centrifuged for 5 minutes at 10000×g. The supernatant is discarded. Bio-Rad cell lysis solution is added. The volume of cell lysis solution varies based on tissue volume. Two times the tissue volume of lysis solution is added. The pellet and lysing solution are vortexed/shaken to re-suspend the pellet. The mixture is placed in the refrigerator and allowed to sit for 5 minutes.

Centrifugation: After one of the above procedures is completed the homogenate is then placed into 250 mL containers and centrifuged at 15000×g for 10 minutes. This step separates the cellular debris from the fluid. The fluid should contain chemokines, cytokines, and growth factors. The supernatant is retained.

Vacuum filtration: The supernatant from the above step is placed in a vacuum filtration system. The system contains two filters. The initial filter has a 1 μm pore size, which is followed by a filter with a 0.45 μm pore size. This step is to remove any remaining cellular debris and to clarify the supernatant. The filtrate is retained.

HPH (human placental homogenate) Preparations-PH can be preserved in one of the following ways: (1) lyophilized and placed in sterile vials with testing to ensure bioactivity following lyophilization: (2) placed in sterile vials in liquid form for later use, for example, for injection: or (3) spray dried. HPH may be combined with other placental products, such as amniotic membrane. In one example, a-soaked amniotic membrane, the following process may be used.

### Example 3: Wound Assay

A scratch assay, a standard in vitro assay of wound healing, was performed on 3 samples of both 1×PBS and 1×PBS-1×PI from a placental disc extraction, generated using the methods described in Example 1. Human skin fibroblasts were cultured in Dulbecco's Modified Eagle's Medium (DMEM) with 10% fetal bovine serum in 12 well plates. When a monolayer was achieved, a cell scraper was used to remove a thin line of cells from the culture bottom. The wells were washed 3 times with Dulbecco's phosphate buffered saline (DPBS) to removed dissolved cells. Test articles were administered in serum-free DMEM at a volume approximately 10% of the total volume in each well. Plates were analyzed on Array Scan ZTI per manufacturer instructions, with photomicrographs taken every 2 hours for the first 24 hours, then once at 48 and 72 hours.

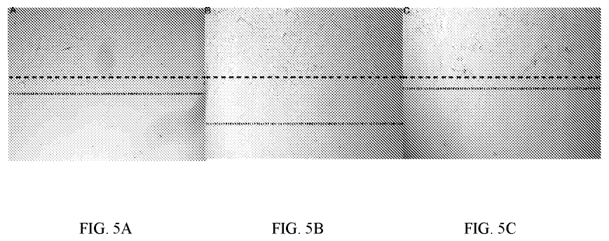

FIGS. 5A-5C show the results of a wound healing assay. A 24-hour In Vitro Wound Healing Assay was conducted where the Fibroblasts were treated with (FIG. 5A) negative control, normal growth media containing no serum, (FIG. 5B) 10% by volume of HPH solution processed with protease inhibitors in the media as described above, or (FIG. 5C) 10% by volume HPH solution processed without protease inhibitors in media described in FIG. 5A. The top line in the images represents the original line of wounding at time 0), while the bottom line indicates the furthest movement of cells into the wounded space. A greater distance is desirable for wound healing. Images are from 24 hours post-injury. The treatment of fibroblast with HPH processed with inhibitor (FIG. 5B) provided nearly twice the cell migration as the negative control (FIG. 5A). The negative control (FIG. 5A) was approximately equal to that of the HPH processed without inhibitors treatment (FIG. 5C).

### Example 4: Quantifying Cytokines and Growth Factors Present in the Placental Preparation

The placenta/amniotic membrane was procured from a contract hospital via a placenta donation. The donor was screened according to American Association of Tissue Banks (AATB). The placenta/amniotic membrane was placed in 0.9% normal saline (NS, 154 mM NaCl, 308 mOsm/L). The placenta was removed from the 0.9% NS, the placental disc was separated from the amniotic membrane, chorion, and umbilical cord, and dissected into approximately 1.5 inch by 2 inch pieces. These smaller pieces of placental disc were placed in a container with approximately 400 mL of 1× phosphate buffered saline (PBS, 137 mM NaCl, 2.7 mM KCl, 4.3 mM Na₂HPO₄, 1.47 mM KH₂PO₄, pH 7.4). The container was placed on a shaking platform shaken at 120 rpm for 12 to 24 hrs. The placental disc pieces were grossly homogenized using a laboratory blender with either 1×PBS or 1×PBS that contained protease inhibitor (PI) at a 1× final concentration (AEBSF 500 uM, Aprotinin 150) nM, E-64 1 uM, Leupeptin 1 uM). The gross homogenate was placed into 250 mL containers and centrifuged for 10 minutes at 4,000× gravity (g). The gross homogenate was poured into the container for a ratio of approximately 1:5, tissue volume: total volume. The container was vortexed/shaken to re-suspend the pellet. The suspension was centrifuged for 10 minutes at 4,000×g. This wash procedure was repeated for a total of three washes. The mixture was not centrifuged after the last wash. The washes remove blood from the tissue. The contents of the 250 mL containers were diluted with either 1×PBS or 1×PBS-1×PI to double the volume. The mixture was then placed into a reservoir feed connected to a high-pressure homogenizer. The homogenate was then placed into 250 mL containers and centrifuged at 15,000×g for 10 minutes. This procedure was performed on 6 donated placental discs.

The full term amniotic fluid was procured from a contract hospital via donation, where the donor was screened according to AATB standards. Fluid from 10 donors was collected for quantification of cytokines and growth factors.

A Bio-Plex MAGPIX Multiplex Reader (Bio-Rad Laboratories, Hercules, CA) was used to quantify the amounts of cytokines and growth factors present in the solution extracted from the placental disc. A Bio-Plex Pro Human Chemokine plane, 40-plex assay (Bio-Rad Laboratories, Hercules, CA) was obtained. The standards were diluted in appropriate diluent. Assay beads were diluted and 50 μL was added to each well of the 96-well plate and washed twice with assay buffer. The plate was placed on a magnetic plate holder and the solution removed from the wells. The standards, samples and blanks were loaded into each respective well of the 96-well plate and incubated for 1 hour at room temperature (note: duplicate measurements were performed on each standard, sample and blank). The plate was washed by placing the plate on the magnetic plate holder, removing the solution, washing with wash buffer, and removing the wash buffer by placing the well plate on the magnetic plate holder. The detection antibodies were added to each well and incubated for 30 minutes at room temperature. The plate was washed 3 times with wash buffer and the streptavidin-PE indicator was added to each well. The plate was incubated for 10 minutes in the dark at room temperature. The plate was washed 3 times in assay buffer and measurements were taken on the MAGPIX multiplex reader. Data was analyzed on Bio-Plex reader software (Bio-Rad Laboratories, Hercules, CA) and a two-way ANOVA was utilized to compare the treatment groups.

The values of cytokines and growth factors from 16-20 week amniotic fluid were obtained from publications: (1) Heikkinen J, Mottonen M, Pulkki K, Lassila O, Alanen A. Cytokine levels in midtrimester amniotic fluid in normal pregnancy and in the prediction of pre-eclampsia. Scand J Immunol. 2001: 53 (3): 310-4: (2) Payne M S, Feng Z, Li S, Doherty D A, Xu B, Li J, Li L, Keelan J A, Zhou Y H, Dickinson J E, Hu Y, Newnham J P. Second trimester amniotic fluid cytokine concentrations, Urea plasma sp colonization status and sexual activity as predictors of preterm birth in Chinese and Australian women. BMC Pregnancy Childbirth. 2014:14:340; and (3) Burns C, Hall S T, Smith R. Blackwell C. Cytokine levels in late pregnancy: Are female infants better protected against inflammation? Front Immunol; 6:318.

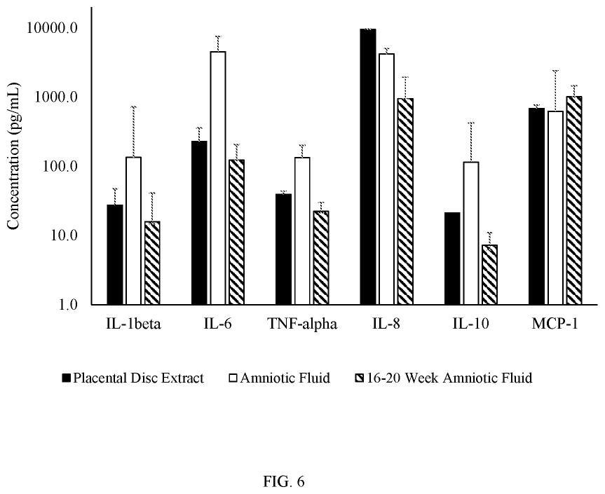

Results are shown in FIG. 6. The methods described produced a less variable product than that of the term amniotic fluid of 16-20 week amniotic fluid, as indicated by the lower standard deviation. The methods described produced a product that varied by 11 to 25%, whereas the quantities in the natural product (both amniotic fluids) can vary from 44 to over 100%. There was also a balance between the inflammatory and anti-inflammatory components of the product produced by the methods described. The inflammatory components, IL-1 beta, IL-6, and TNF-alpha, were lower in the product produced by the described methods than those found in term amniotic fluid. There was a more favorable ratio of IL-6 to IL-10 in the product described by the method (10.8) than in term amniotic fluid (34.5). There were higher quantities of anti-inflammatory components, IL-8 and MCP-1, in the product produced by the described method than those found in term amniotic fluid. The quantities of these proteins, IL-1 beta, IL-6, TNF-alpha, IL-8, IL-10, and MCP-1, in the product produced by the methods described was more similar to the quantities reported for 16-20 week amniotic fluid than term fluid.

### Example 5: Placenta Pooling

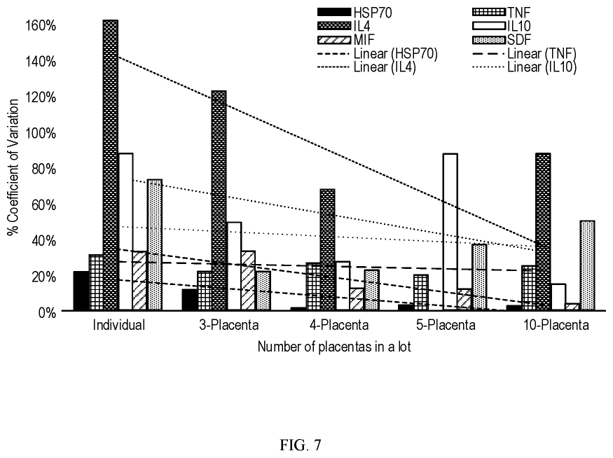

Pooling can lower protein concentration variability with more placentas per manufacturing lot (FIG. 7). For pooling, ten placentas were obtained from the American Association of Tissue Banks (AATB), a FDA registered tissue bank. Tissues were processed to remove maternal and fetal blood and frozen at −80° C. Placentas were thawed and aliquots were removed to process each individually, generate three lots containing three placentas, generate three lots containing four placentas, generate three lots containing five placentas, with the remaining material pooled into a single lot containing all ten placentas. Protein levels were adjusted to make the lots contain equivalent amounts of protein. Individual proteins Heat Shock Protein 70 (HSP70), tumor necrosis factor alpha (TNF), Interluekin (IL)-4 (IL4), IL10, Macrophage Inhibitory Factor (MIF), and SDF-1 were quantified by protein specific ELISA. Results shown in FIG. 7 are shown as percent (%) coefficient of variation.

### Example 6: Osteoarthritis (OA) Related Proteins within the Methods Described

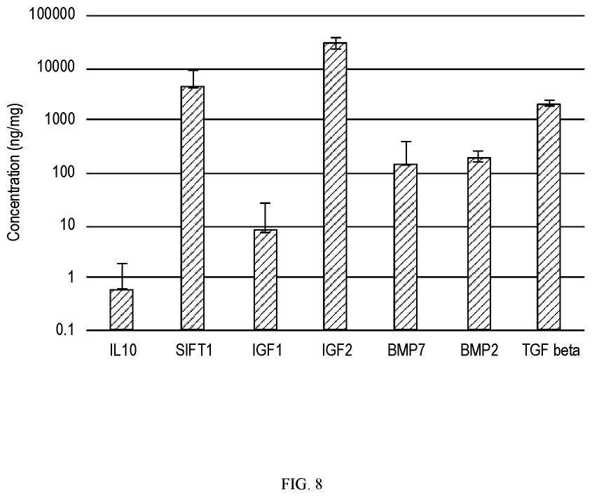

Several proteins present in the placental composition may interfere with osteoarthritis (OA) mechanisms and reduce OA severity as shown in FIG. 8. Ten placentas were obtained from AATB, FDA registered tissue banks. Tissue was processed as described in Example 5, pooling the 10 placenta once donors were cleared and before the final cell lysis step. Following, the final product was rehydrated and individual proteins were quantified using a Ray Biotech KiloPlex Assay (Norcross, GA). FIG. 8 shows data reported as nanogram (ng) of individual protein per milligram (mg) of total protein present in the mixture.

### Example 7: Radiation and Arthritic-Changes in Non-Human Primate Joints

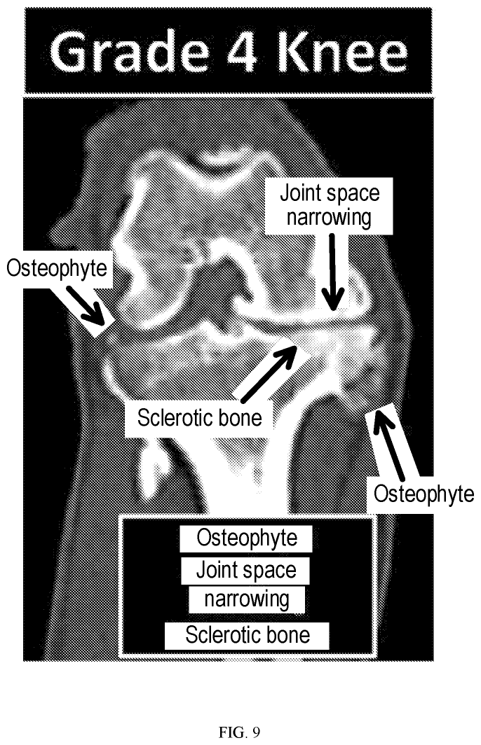

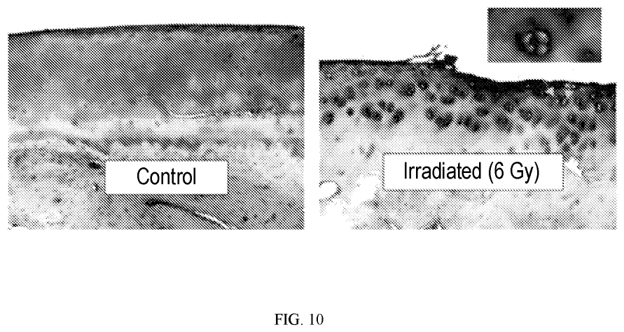

A study of a Radiation Late Effects Cohort (RLEC) of non-human primates (NHPs) showed that 67% of the cohort exhibited radiographic OA after long-term radiation toxicity exposure. FIG. 9 shows a radiographic osteoarthritis X-Ray from the knee of a NHP from the RLEC. Preliminary histologic data from the femoral heads of irradiated NHPs indicated that articular cartilage lining the hip joint exhibits: loss of GAGs: fibrillation; and chondrocyte clustering, which all represent arthritic phenotypes (FIG. 10) after long-term radiation toxicity exposure. Clustering of chondrocytes, in particular, is a hypertrophic response.

### Example 8: Placental Preparations and Arthritic Responses In Vitro

Matrix metalloproteinase 13 (MMP-13) and ADAMTS5 (ADAM Metallopeptidase with Thrombospondin Type 1 Motif 5) may contribute to the progression of arthritis by degrading type II collagen and GAGs.

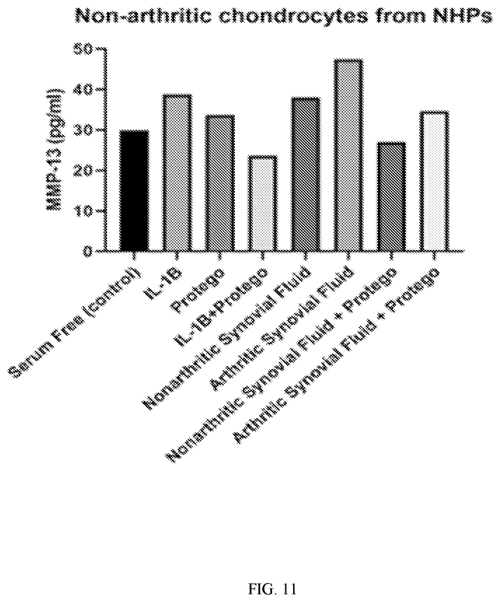

MMP-13 production from non-human primate (NHP) chondrocytes in 2D culture was increased when the chondrocytes were treated with either: IL-1β: or knee joint synovial fluid collected from NHPs of the RLEC that exhibited arthritis in the knee (FIG. 11). The placental preparation may prevent this increase in MMP-13 release from chondrocytes.

### Example 9: Radiation and Development of Arthritis in Rodent Joints

It has been found that exposing rats to survivable doses of radiation can lead to degradation of cartilage and may lead to loss of type II collagen, reduced glycosaminoglycan (GAG) content, and arthritis in the knee joints. The loss of articular cartilage or its constituent type II collagen and GAG content may be associated with the development of symptomatic arthritis. For example, rats receiving 1, 3, or 7 Gy of TBI exhibit arthritis of the knee in a dose-dependent manner nine months after the exposure. Even a 1 Gy dose (10 MV X-rays) results in an acute loss of type II collagen and GAG content in rat knees at 13 days post-irradiation. Despite the low 1 Gy acute exposure, elevated matrix metalloproteinase 13 (MMP-13) activity, chondrocyte clustering, and degraded type II collagen, arthritic responses within the knees of rats, persists out to seven weeks post exposure. In another example, in mouse models, doses as low as 0.1 Gy TBI (10 MV X-rays) results in arthritic changes in the knee articular cartilage after 24 days, including: (1) thinning of the cartilage: (2) elevated MMP-13 activity: (3) loss of cartilage volume; and (4) loss of type II collagen. It has been found that proteomic assessment of the articular cartilage lining the femoral head measured loss of matrix components, including ColXα1, aggrecan core protein and chondroadherin, among others. Alterations in signaling pathways also indicated that the degradation of the cartilage occurred with pro-arthritic signaling patterns, including reduced anabolic mTOR signaling, increased oxidative stress-mediated pathways (e.g., altered NRF2 signaling), and increased cell death pathways. Degradation was ongoing on day 24 post exposure, as serum cartilage oligomeric matrix protein (sCOMP) was elevated, indicating active breakdown of cartilage. Thus, in vivo pre-clinical models indicate that even low doses of radiation result in persistent arthritic responses in cartilage in joints.

### Example 10: Radiation and New Matrix Formation

In vivo studies indicated that anabolic mammalian target of rapamycin (mTOR) signaling may be reduced in the femoral head articular cartilage of mice after TBI. In vitro data supports these measurements as GAG synthesis in cartilage may be lower after irradiation. The loss of matrix production may be related to a dose-dependent decrease in Akt activation, which may not be prevented by anabolic IGF-1 stimulation. Akt activation may be required for normal articular cartilage formation and homeostasis. The in vitro and in vivo data indicate radiation induces an osteoarthritic phenotype in chondrocytes and promotes the development of arthritis.

### Example 11: Radiation and Hypertrophic Phenotype in Chondrocytes

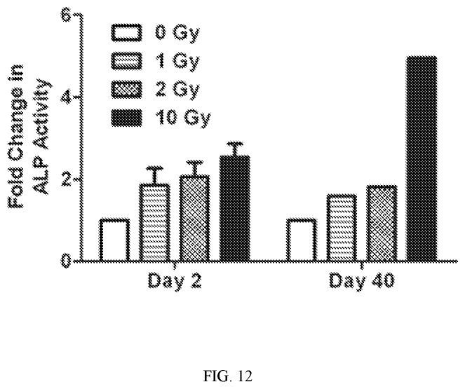

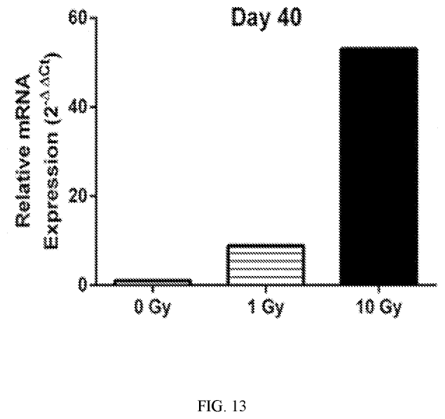

ALP activity (FIG. 12) and type X collagen expression (FIG. 13) were increased in pig chondrocytes at Day 2 (ALP) and 40 (ALP and type X collagen) after exposure to doses up to 10 Gy. the increase in ALP activity after irradiation may impact joint calcification.

### Example 12: Radiation and Hypertrophy-Mediating Wnt Signaling

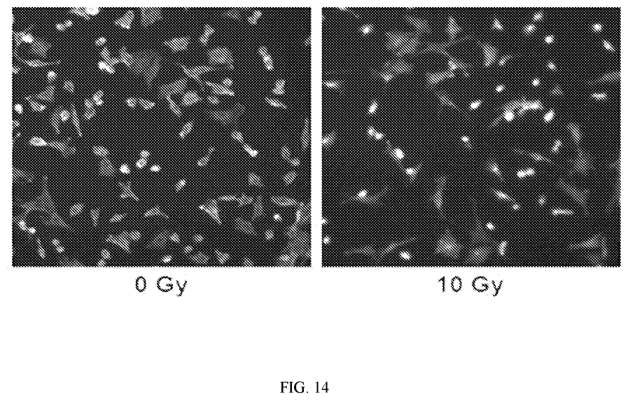

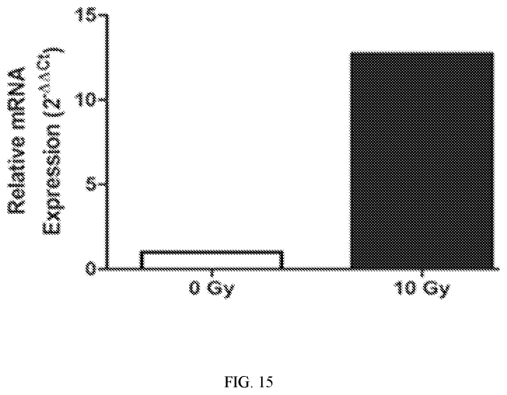

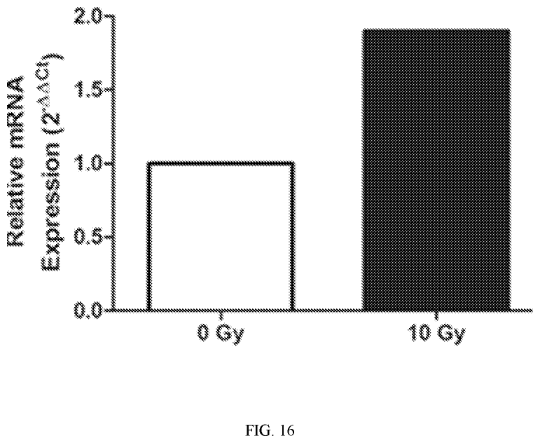

In vitro studies with pig chondrocytes demonstrated that nuclear β-catenin levels (FIG. 14) and expression of RUNX2 (FIG. 15) are increased from Day 1 after a high non-survivable, 10 Gy dose of radiation. Expression of LEF-1 in human chondrocytes also increased from Day 1 after a high non-survivable 10 Gy dose of radiation (FIG. 16). LEF-1 has been implicated in cartilage degradation during arthritis.

### Example 13: Placental Preparation and Osteoarthritis in a Rat Model of Traumatic Injury

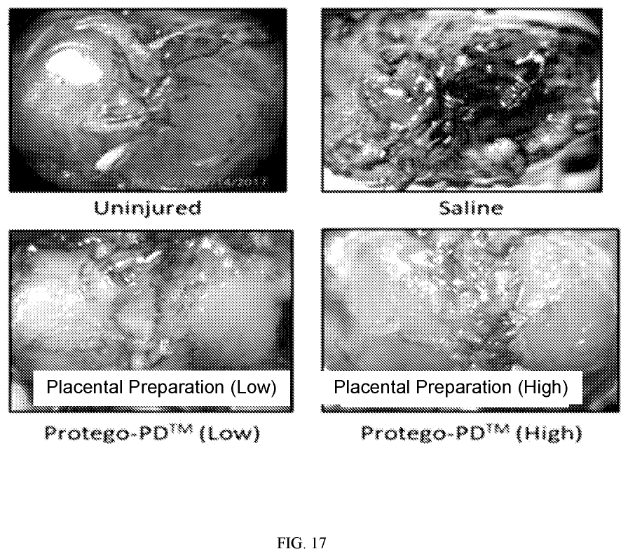

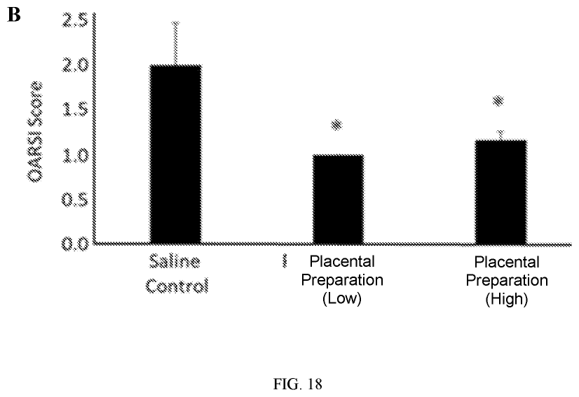

A severe rodent joint injury model of post-traumatic osteoarthritis (PTOA) was used to determine the impact of the placental preparation on the development of PTOA. Knee joint damage was induced in Lewis rats via transection of the medial cruciate (MCL) and cranial cruciate ligaments (CCL) and rarefication of the medial meniscus (MM). Treatment with either normal saline or 7.7 mg protein/mL or 38.5 mg protein/mL of the placental preparation in normal saline was administered via a single intra-articular injection at the time of injury. Four weeks post-injury gross morphological cartilage lesions were observed and scored by three blinded reviewers. Cartilage lesions in the 38.5 mg/mL treatment were significantly decreased from the saline-treated controls (p<0.05). No significant differences were observed between the 7.7 mg/mL dose of the placental preparation and the saline control or between the 7.7 mg/mL and 38.5 mg/mL of the placental composition with protease inhibitor treatments. FIG. 17 shows images of the uninjured morphological cartilage of a rodent knee as well as injured morphological cartilage for cartilage induced via transection of the MCL and CCL and rarefication of the MM when treated with saline, low concentrations of placental preparation (a 7.7 mg/ml dose), and high concentrations of placental preparation (a 38.5 mg/mL dose). FIG. 18 illustrates a graphical representation of the OARSI Score for each of the treated conditions for the injured rodent knee and shows the significant impact of treated knee joint versus the saline control.

### Example 14: The Placental Preparation and the Release of Cartilage-Degrading MMP-13, MMP-1, and GAGs from IL-1-Treated Cartilage In Vitro

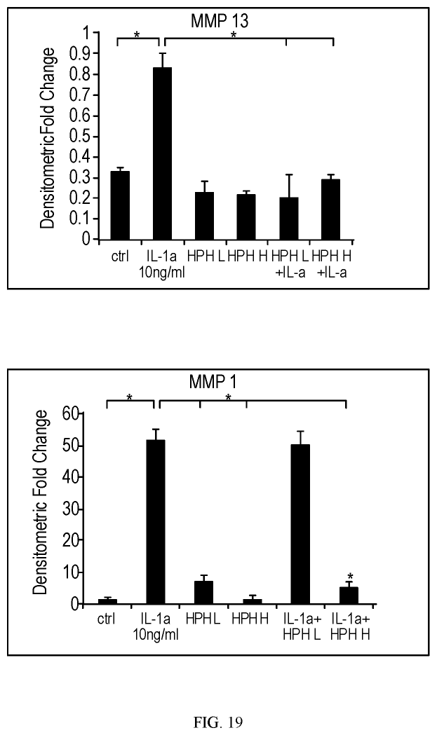

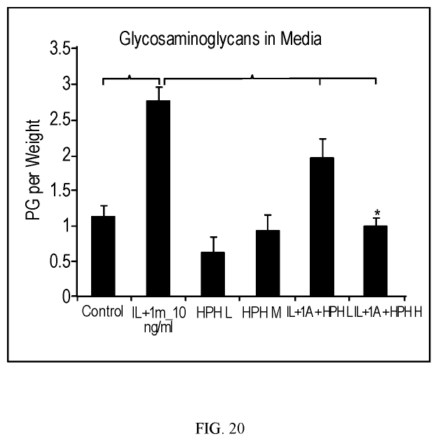

Primary porcine chondrocytes (high-density monolayer cultures) and 6-mm cartilage explants were stimulated IL-1α (10 ng/ml) in the presence and absence of the placental preparation (3.75 mg/ml (low dose-HPHL) or 5.625 mg/ml (high dose-HPHH)) for 48 hours. As illustrated in FIG. 19, the placental preparation appears to have prevented IL-1α-mediated increases in MMP-1 and MMP-13 secretion and from porcine chondrocytes were not observed. FIG. 20 shows that the placental preparation IL-1α mediated increase in GAG secretion were not observed. Similar results were seen in human arthritic chondrocytes and meniscal cells collected at total arthroplasty procedures for terminal osteoarthritis: the placental preparation (5.625 mg/ml) reduced IL-1β-induced increase in MMP-1, MMP-3, MMP-8, MMP-9) and MMP-13 production.

### Example 16: Prophetic Example to Determine the Severity of Osteoarthritis (OA) in Rats with ACL Transection (ACLT) and/or TBI Exposure

To detect if an arthritic response is enhanced with both traumatic injury and exposure to radiation and, for examining long-term changes, there is a need to be able to discriminate if each challenge generates arthritic responses individually, and at the lowest dose for which there is preliminary/published data. A 1 Gy dose of total body irradiation (10 MV X-rays) resulted in an 89% (SD 35%) increase in MMP-13 and a 112% (SD 41%) increase in VDIPEN (an epitope for MMP-degraded collagen) present within the knees of adult rats by seven weeks after exposure: these occurred coincident with increased chondrocyte clustering, and overall arthritic responses throughout the knees on MRI imaging. Given the similarities of the magnitude difference in these two related biomarkers, MMP-13 data will be used as the basis for the analyses. With 20 groups (ACLT or SHAM: 4 doses of radiation or SHAM: 2 time points), using a 2 Way ANOVA (main effects PTOA and Injury); a sample size of 8 is sufficient to discriminate differences at a desired power of 80% and accepting α=0.05.

Rats will receive either ACLT or anesthesia prior to surgical procedures. ACLT has been shown to induce rapid arthritic changes in the knees of rats. The rats will be lightly anesthetized with isofluorane. A 5-10 mm medial parapatellar knee joint incision will be created in both the right and left knee joints. The ACL will be transected and a section will be removed. Underlying tissues will be sutured with absorbable 6-0 vicryl sutures using an interrupted square knot with 3-4 throws. Staples will be used for the outer exposed skin and removed 7-10 days after surgery. NS rats will be lightly anesthetized with isofluorane. No incision will be made.

The TBI rats will be lightly anesthetized with isofluorane, and whole body irradiated for 4 consecutive days with 3 Gy/day of 10 MV X-rays using a clinical linear accelerator (LINAC). No anesthesia will be provided: the rats will be placed into a custom device that permits the TBI of rats without anesthesia. The rats will be placed into a horse compression sock, which will calm the rats and prevents motion as they are placed into the apparatus and irradiated. In previous experiments, no movements were observed at doses as high as 10 Gy delivered over 20 minutes from rats. Radiation will be delivered in two laterally opposed beams: dosimetry will be provided with the LINACs.

Prior to sacrifice, blood will be collected from each rat via cardiac puncture while under heavy isofluorane anesthesia. Serum will be isolated by centrifugation and stored at −80° C. until analysis. Serum will be collected every 2 months in order to assess ongoing cartilage breakdown using serum COMP as a circulating biomarker. After sacrifice, the right and left hind limbs will be removed by displacing the femoral heads from the acetabulum. The femur will be cut just proximal to the femoral head, and the cartilage from the femoral head will be collected using a mini-Rongeur for molecular assessment after tissue digestion for lysates. The right knee will then be fixed in 10% neutral buffered formalin. After 3 days of fixation, the right hind limb will be switched to 70% EtOH. The right knee will be analyzed using 7T MRI, and histological assays. The femur and tibia of the left hind limb will be disarticulated. The articular cartilage lining the 1) femoral condyles, 2) tibial plateau; and 3) femoral head will then be removed with a mini-Rongeur to directly measure loss of GAGs (DMB assay) and to perform gene expression panels, alkaline phosphatase activity, and collagen content (hydroxyproline).

A total of 160 male Lewis rats will be entered for the study (Table 3). These rats will be purchased at 15 weeks old, acclimated for one week, and then divided into two groups. Group 1 will receive a surgical procedure on the knee known to cause arthritis (ACLT). Group 2 will receive no surgical procedure (NS).

TABLE 3
Tissue Collection Schedule

| Group | 6 Month | 12 Month |
|---|---|---|
| ACLT | | |
| SHAM | 8 | 8 |
| 0.5 Gy | 8 | 8 |
| 1.0 Gy | 8 | 8 |
| 3.0 | 8 | 8 |
| 5.0 | 8 | 8 |
| NS | | |
| SHAM | 8 | 8 |
| 0.5 Gy | 8 | 8 |
| 1.0 Gy | 8 | 8 |
| 3.0 | 8 | 8 |
| 5.0 | 8 | 8 |

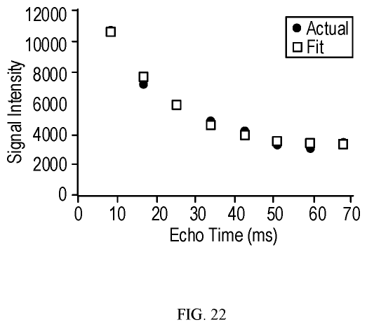

Blood will be collected immediately prior to surgical procedures for serum isolation, and then every two months until the end of the study. Four months after the surgery, rats from each surgical group will be subdivided (Table 3) and exposed to survivable TBI doses of 0 (SHAM), 0.5, 1, 3, or 5 Gy. Unirradiated rats from each surgical group will serve as controls. At both 6 (2 months post-irradiation) and 12 months (8 months post irradiation) after the surgical procedures, the hind legs will be collected and articular cartilage degradation, altered molecular composition of knee joint tissues and chondrocyte hypertrophy will be assessed using, (1) 7T MRI microimaging to assess T2 relaxation times for joint degradation and overall matrix changes (FIG. 22): (2) biochemical techniques that include quantifying GAG content loss (dimethylmethlene blue assay: DMB: (FIG. 18) collagen content (hydroxyproline colorimetric assay, as previously described by us; and mineralization (alkaline phosphatase activity, ALP, (FIG. 12): (3) histologic and immunohistochemical techniques employed by our lab to quantify arthritis (H&E), proteins associated with changes in matrix GAG content and collagen content (Movat Pentachrome), cartilage degradation (MMP-13), synovial inflammation (IL-1B), and hypertrophy (e.g., collagen type X; β-catenin as a biomarker for Wnt activity and hypertrophy): (4) serum ELISA for cartilage oligometric matrix protein (sCOMP) to potentially identify a biomarker for cartilage degradation, (93-95); and (5) mRNA expression associated with both normal (Col2a1, ACAN) and hypertrophic (Runx2, Col10a1, MMP13, ADAMTS5, and LEF1) chondrocyte genes will be quantified using RT-qPCR with TaqMan primer/probe sets (Applied Biosystems, Foster City, CA): (FIGS. 13, 15, and 16).

It is expected that cartilage degradation will not be observed in rats that received no surgery and no irradiation. It is expected that ACLT alone to cause degradation of cartilage in rat knees observed at both 6 and 12 months after surgery. Likewise, it is expected that TBI will cause degradation of knee joint articular cartilage at 2 months post irradiation that will become more severe at 8 months post irradiation. It is expected that radiation induced articular cartilage degradation will be dose dependent. Finally, it is expected that articular cartilage lining the tibial plateaus will be more degraded and with a greater degree of inflammation after receiving both ACLT and TBI relative to controls at 6 months, and that this damage will be progressively worse at 12 months after ACLT. It is expected that biomarkers of OA, inflammation, and chondrocyte hypertrophy will be present in the irradiated knees. It is expected that T2 relaxation times will be greater, indicating degraded GAG content, in the cartilage from rat knees that are exposed to radiation+ACLT than either injury individually.

### Example 17: Prophetic Example to Determine if the Placental Preparation can Reduce Established Knee Arthritis and Cartilage Degradation after ACLT and/or TBI

With traumatic injury, the placental composition lowers Osteoarthritis Research society International (OARSI) scores by 37% (SD 16%). Power estimates will be based on PTOA data. As such, given 12 groups (ACLT or NS: 1 dose radiation (IR) or SHAM irradiation (0) Gy): 2 doses of the placental preparation or vehicle (VEH)) using a 2 Way ANOVA (main effects PTOA and Radiation): a sample size of 8 will be sufficient to discriminate differences at a desired power of 80% and accepting α=0.05.

Blood will be collected immediately prior to surgical procedures for serum isolation, and then every two months until the end of the study to assess biomarkers of knee degradation/prevention longitudinally. Rats will receive either ACLT or NS. After 4 months of recovery from the surgery, we will subdivide the ACLT and NS rats (Table 4) and then) expose the rats to the lowest dose of radiation shown to induce arthritis from Example 15: or 2) if there was no additional damage from the radiation from Example 16, use the highest survivable dose of radiation that was applied. The day after the TBI is performed, rats from each group will be subdivided and receive either one of two doses of the placental preparation or the vehicle control via intraarticular injections. In our initial pilot experiment (FIG. 16), the placental preparation was administered in a single 0.1 mL injection at a total protein concentration of 7 mg/mL and 35 mg/mL. That equates to a 2.5 mg/kg and 12.5 mg/kg dosage based on their body weight at the time of surgery. These single doses at the time of injury provided durable protection at 4 weeks. A multiple injection format will be used to assess the impact of the placental preparation on OA remote from the initiating injury over a longer period of potential progression complicated by a second injury via radiation exposure. Rats will receive a series of 3 monthly injections of the placental preparation beginning the day following radiation exposure. Progression of the knee damage will be characterized over a 1-year period with similar approaches as in Example 16.

It is expected that cartilage degradation will not be observed in rats that received no surgery and no irradiation. It is expected that ACLT alone to cause degradation of cartilage in rat knees, and that TBI to cause degradation of cartilage at 8 months post irradiation. It is expected that sCOMP levels to worsen in rats receiving ACLT and/or IR throughout the study, with no change in the NS or SHAM groups. It is expected that combined ACLT and IR to worsen the overall degree of OA from all assays, with associated elevation in inflammation in the joint. Not intending to be bound by theory, the placental preparation may mitigate this increased serum biomarker of cartilage degradation in treated rats from the ACLT and/or IR groups. The placental preparation may reduce the overall degree of OA as measured histologically and from 7T MRI, with T2 relaxation times indicating a mitigation of the loss of GAGs or matrix relative to ACLT and/or IR groups that did not receive the placental preparation. Biomarkers of OA (including MMP-13, vDIPEN, increased ALP, and hallmarks of hypertrophy including chondrocyte clustering, calcifications, and nuclear β-catenin) may be reduced in placental preparation treated ACLT and IR exposed rats versus non-treated. T2 relaxation times may be greater in the cartilage from rat knees that are exposed to radiation+ACLT than either factor independently, indicating degraded GAG (a biomarker of extracellular matrix) content.

### Example 18: Prophetic Example to Determine if the Placental Preparation can Protect Against Bone Loss

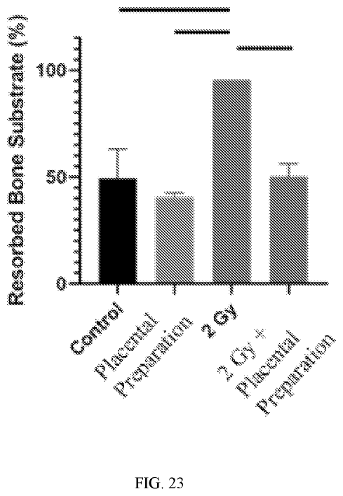

Example 13 indicates that the placental preparation can mitigate OA in a PTOA injury model and also reduce MMP-13 production in a radiation model. It is possible that the placental preparation will not reduce phenotypic OA from 1-dose radiation (IR) rats, if a radiation-induced mechanism causing the damage is not one that can be reversed by the agent (e.g., hypertrophy). In such a case, the placental preparation will be examined to see if it can protect against bone loss in those bones from the MRI scans and the measurement approach using Mimic's software. Radiation causes acute and persistent bone loss in rodent, NHPs, and human clinical conditions. Pathologic fractures would represent a substantial source of morbidity in survivors of a radiologic event. Data indicates that the placental preparation reduces radiation-induced osteoclast activity (FIG. 23), which has been identified as a primary cause for pathologic bone loss caused by TBI. Whether the placental preparation protects against radiation-induced cartilage degradation, bone responses from these rats will be measured to identify if the placental preparation is a countermeasure to prevent radiation induced bone loss and consequent fractures.

### Example 19: Determining if the Placental Preparation can Reduce Arthritis and Cartilage Degradation in Knees of NHPs Exposed to Ionizing Radiation

Rhesus macaques from the Radiation Late Effects Cohort (RLEC), a unique group of previously irradiated NHPs at Wake Forest University (WFU) will be used. This cohort consists of a running total of ˜200 male and female rhesus monkeys. The RLEC is the first population of long-term NHP radiation exposure survivors to be characterized at a high level of clinical and pathologic detail since the advent of modern molecular biologic techniques, in which “omics” can be linked to disease phenotypes and tissue responses. The longitudinal value of the dataset is unparalleled: animals have been observed for of 4.6+/−3.1 years, with the longest survivors now 15 years out from radiation exposures. Daily care and observations, routine clinical blood count and clinical chemistry data encompass>750 monkey-years and over 28,000 episodes of animal contact, resulting in over 120,000 basic clinical pathology data points. Additional datasets include flow cytometric immune profiling, peripheral blood mononuclear cell gene expression, and exome sequencing. Imaging data includes >700 annual CT scans>600 DEXA bone density scans, >800 echocardiograms, and >400 3T MRI brain scans. Recently added exome sequencing and MHC SNP typing on most of the current animals in the cohort, have increased the volume and inferential scope of the dataset exponentially.

In addition to what is discussed in Example 7, from the entire cohort, 10 control and 28 irradiated NHPs exhibit bilateral OA of the knee; it is from these NHPs that n=16 NHPs will be selected for enrollment into this proposed project, n=8 irradiated and n=8 non-irradiated controls. One arthritic knee joint from each irradiated and control NHPs will be treated with the placental preparation injected directly into the referent joint (with the opposite arthritic joint serving as an internal control). The protective effect of the placental preparation on cartilage health will be determined six months post injection. Any systemic effects will be detected by analyzing effects in both non-treated irradiated and non-irradiated control NHPs.

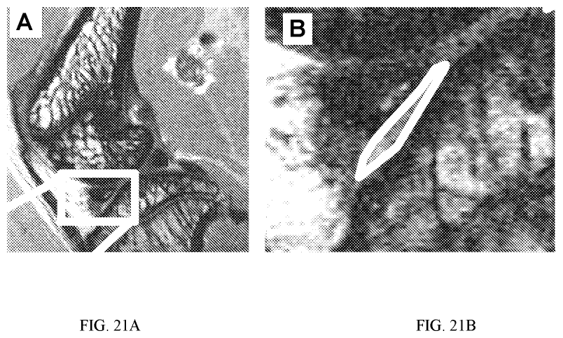

The mean degrees of OA (Kellgren-Lawrence grading) for both knees from the SHAM NHPs is 3, and from the IR NHPs is 2, thus the degrees of OA between groups and knees are both similar and not extreme. Treatment with the placental preparation will only be delivered to one knee (RIGHT), with the left knee serving as the internal control. NHPs from both the SHAM and IR groups will be randomly divided into groups (FIG. 21) to either receive a 12.5 mg/kg dose of the placental composition in each of 3 separate 1 mL intraarticular injection into the RIGHT knee, or a corresponding VEH (0.9% NaCl: saline) intraarticular injection: this dose, delivered monthly for three months is designed to overcome the chronic processes that the animals have been exposed to, including aging. Animals will receive 3 monthly injections, as is common practice with steroids, hyaluronic acid, platelet-rich plasma, and amnion-based products in humans. Blood for serum isolation will be collected at the outset of the study from NHPs, and then every two months until the tissue harvest. Synovial fluid will be collected from both knees at the outset of the study, at 3 months, and then at tissue harvest. Computed tomography (CT) scans will be performed using the onsite CT scanner at the RLEC housing facility. A baseline CT scan will be performed at the outset of the study, and then two, four, and six months thereafter.

Progression and reversal of the existing, radiographically documented knee damage will be characterized over a 6 month period that involves longitudinal, bi-monthly assessments of: 1) clinical signs, including mobility measured by video monitoring and knee kinematics, including range of motion by goniometry: 2) radiographic OA as determined from CT scans, with knee OA graded using the Kellgren-Lawrence grading scale (FIG. 9): 3) hematology and serum chemistry (clinical panel) to monitor animal health and systemic indicators of inflammation: 4) direct measures of cartilage breakdown, ongoing arthritic status, and general inflammation within the knee joint capsule via GAG, MMP-1, MMP-13, ADAMTS5, and IL-1β quantification from synovial fluid: 5) sCOMP to estimate the degree of overall cartilage breakdown and: at 3 months and tissue harvest: synovial fluid and at tissue harvest: decalcified histology of the isolated right and left knee joints in order to quantify arthritis (H&E) through the joint as an organ (including the articular cartilage, menisci, and synovium), proteins associated with changes in tissue matrix and collagen content (Movat Pentachrome), cartilage and meniscal degradation (MMP-1, MMP-13, ADAMTS5, and vDIPEN), and hypertrophy (e.g., Col X and chondrocyte clustering); and 6) overall cartilage and meniscal degradation as determined using T2 relaxometry from 7T MRI (FIG. 9). Clinical examinations will include twice-daily observation for mobility, abnormal gait, or signs of discomfort; and weekly to monthly physical exams under ketamine+dexmedetomidine sedation to assess ROM bilaterally and assess for changes in joint temperature by infrared thermometry, swelling, erythema, crepitus or other clinical abnormalities. Synovial fluid will be assessed cytologically at each time point collected, for any evidence of inflammation or infection. Analgesics (buprenorphine and/or ketoprofen) will be used for any animals showing signs of pain.

At the study's end, all animals will be euthanized by pentobarbital anesthesia followed by exsanguination, perfusion with saline, and immediate collection of target joints and a full necropsy including gross and histopathologic assessment of tissues from all major organ systems.

For the 6-month study period, it is expected that OA progression will remain stable or worsen in NHPs in both the VEH and SHAM groups. It is expected that a greater degree of hypertrophy in the OA observed in the knees from IR NHPs than in SHAM irradiated NHPs. It is expected that the effect of the placental preparation is local to the treated joint, such that intraarticular injection of the placental preparation may reverse the severity of OA compared to both the non-treated, contralateral knee of the same NHP/within groups and compared to the joints from VEH-treated NHPs. It is expected that the improvements in OA in the placental preparation-treated knee joints may be identifiable by reduced Kellgren-Lawrence scores from CT scans, from improved histologic outcomes of OA and inflammation, from lower sCOMP, from improved mobility and ROM, and from increased T2 relaxation times as measured from 7T MRI.

### Illustrative Embodiments of Suitable Compositions and Methods

As used below, any reference to compositions, articles, or methods is understood as a reference to each of those compositions, articles, or methods disjunctively (e.g., “Illustrative embodiment 1-4 is understood as illustrative embodiment 1, 2, 3, or 4.”).

Illustrative embodiment 1 is a method of treating a condition affecting bone tissue, muscle tissue, or connective tissue using a placenta-derived composition comprising placental tissue and one or more protease inhibitors.

Illustrative embodiment 2 is the method of any preceding or subsequent illustrative embodiment, wherein the placental tissue comprises a placental disc.

Illustrative embodiment 3 is the method of any preceding or subsequent illustrative embodiment, wherein the placental tissue is acellular.

Illustrative embodiment 4 is the method of any preceding or subsequent illustrative embodiment, wherein the one or more protease inhibitors comprises 4-(2-Aminoethyl) benzenesulfonyl fluoride hydrochloride (AEBSF), Bovine Lung Aprotinin, N-(trans-Epoxysuccinyl)-L-leucine 4-guanidinobutylamide, Leupeptin, N-ethylmaleimide (NEM), phenylmethylsulfonylfluoride (PMSF), ethylenediamine tetraacetic acid (EDTA), ethylene glycol-bis-(2-aminoethyl (ether) NNN′N′-tetraacetic acid, ammonium chloride, boceprevir, danoprevir, narlaprevir, telaprevir, or vaniprevir.

Illustrative embodiment 5 is the method of any preceding or subsequent illustrative embodiment, wherein the composition further comprises a diluent, an excipient, a carrier, or combinations thereof.

Illustrative embodiment 6 is the method of any preceding or subsequent illustrative embodiment, wherein the muscle tissue comprises smooth muscle tissue.

Illustrative embodiment 7 is the method of any preceding illustrative embodiment, wherein the connective tissue comprises cartilage, tendons, ligaments, or combinations thereof.

Illustrative embodiment 8 is a method of treating an individual having a malady comprising arthritis, osteoporosis, or fibrosis with a placenta-derived composition.

Illustrative embodiment 9 is the method of any preceding or subsequent illustrative embodiment, wherein the placenta-derived composition comprises a placental tissue and one or more protease inhibitors.

Illustrative embodiment 10 is the method of any preceding or subsequent illustrative embodiment, wherein the placental tissue comprises a placental disc.

Illustrative embodiment 11 is the method of any preceding or subsequent illustrative embodiment, wherein the placental tissue is acellular.

Illustrative embodiment 12 is the method of any preceding or subsequent illustrative embodiment, wherein the one or more protease inhibitors comprises 4-(2-Aminoethyl) benzenesulfonyl fluoride hydrochloride (AEBSF), Bovine Lung Aprotinin, N-(trans-Epoxysuccinyl)-L-leucine 4-guanidinobutylamide, Leupeptin, N-ethylmaleimide (NEM), phenylmethylsulfonylfluoride (PMSF), ethylenediamine tetraacetic acid (EDTA), ethylene glycol-bis-(2-aminoethyl (ether) NNN′N′-tetraacetic acid, ammonium chloride, boceprevir, danoprevir, narlaprevir, telaprevir, or vaniprevir.

Illustrative embodiment 13 is the method of any preceding or subsequent illustrative embodiment, wherein the composition further comprises a diluent, an excipient, a carrier, or combinations thereof.

Illustrative embodiment 14 is the method of any preceding or subsequent illustrative embodiment, wherein the malady comprising arthritis is osteoarthritis.

Illustrative embodiment 15 is the method of any preceding illustrative embodiment, wherein the placenta-derived composition comprises IL-10, SIRT1, IGF1, IGF2, BMP2, BMP7, TGF-β, or combinations thereof.

Illustrative embodiment 16 is a method of treating an individual having irradiation damage to bone, muscle, or connective tissue with a placenta-derived composition.

Illustrative embodiment 17 is the method of any preceding or subsequent illustrative embodiment, wherein the placenta-derived composition comprises a placental tissue and one or more protease inhibitors.

Illustrative embodiment 18 is the method of any preceding or subsequent illustrative embodiment, wherein the placental tissue comprises a placental disc.

Illustrative embodiment 19 is the method of any preceding or subsequent illustrative embodiment, wherein the placental tissue is acellular.

Illustrative embodiment 20 is the method of any preceding or subsequent illustrative embodiment, wherein the one or more protease inhibitors comprises 4-(2-Aminoethyl) benzenesulfonyl fluoride hydrochloride (AEBSF), Bovine Lung Aprotinin, N-(trans-Epoxysuccinyl)-L-leucine 4-guanidinobuty lamide, Leupeptin, N-ethylmaleimide (NEM), phenylmethylsulfonylfluoride (PMSF), ethylenediamine tetraacetic acid (EDTA), ethylene glycol-bis-(2-aminoethyl (ether) NNN′N′-tetraacetic acid, ammonium chloride, boceprevir, danoprevir, narlaprevir, telaprevir, or vaniprevir.

Illustrative embodiment 21 is the method of any preceding illustrative embodiment, wherein the composition further comprises a diluent, an excipient, a carrier, or combinations thereof.

It is contemplated and therefore within the scope of the present disclosure that any feature that is described above can be combined with any other feature that is described above (even if those features are not described together). It should be understood that the present disclosure contemplates and it is therefore within the scope of the disclosure that any element that is described can be omitted from the compositions (except for those necessary to produce the desired utility of the invention) and/or methods of the present disclosure. When ranges are disclosed, it should be noted that any numeric point that fits within the scope of that range is contemplated as a new endpoint for a subrange. Moreover, it should be understood that the present disclosure contemplates minor modifications that can be made to the compounds, compositions and methods of the present disclosure. In any event, the present disclosure is defined by the below claims, which follow and the breadth of interpretation which the law allows.

1. A method of treating a condition affecting bone tissue, muscle tissue, or connective tissue using a placenta-derived composition comprising placental tissue and one or more protease inhibitors.

2. The method of claim 1, wherein the placental tissue comprises a placental disc.

3. The method of claim 1, wherein the placental tissue is acellular.

4. The method of claim 1, wherein the one or more protease inhibitors comprises 4-(2-Aminoethyl) benzenesulfonyl fluoride hydrochloride (AEBSF), Bovine Lung Aprotinin, N-(trans-Epoxysuccinyl)-L-leucine 4-guanidinobutylamide, Leupeptin, Nethylmaleimide (NEM), phenylmethylsulfonylfluoride (PMSF), ethylenediamine tetraacetic acid (EDTA), ethylene glycol-bis-(2-aminoethyl (ether) NNN′N′-tetraacetic acid, ammonium chloride, boceprevir, danoprevir, narlaprevir, telaprevir, or vaniprevir.

5. The method of claim 1, wherein the composition further comprises a diluent, an excipient, a carrier, or combinations thereof.

6. The method of claim 1, wherein the muscle tissue comprises smooth muscle tissue.

7. The method of claim 1, wherein the connective tissue comprises
cartilage, tendons, ligaments, or combinations thereof.

8. A method of treating an individual having a malady comprising arthritis, osteoporosis, or fibrosis with a placenta-derived composition.

9. The method of claim 8, wherein the placenta-derived composition comprises a placental tissue and one or more protease inhibitors.

10. The method of claim 9, wherein the placental tissue comprises a placental disc.

11. The method of claim 9, wherein the placental tissue is acellular.

12. The method of claim 9, wherein the one or more protease inhibitors comprises 4-(2-Aminoethyl) benzenesulfonyl fluoride hydrochloride (AEBSF), Bovine Lung Aprotinin, N-(trans-Epoxysuccinyl)-L-leucine 4-guanidinobutylamide, Leupeptin, Nethylmaleimide (NEM), phenylmethylsulfonylfluoride (PMSF), ethylenediamine tetraacetic acid (EDTA), ethylene glycol-bis-(2-aminoethyl (ether) NNN′N′-tetraacetic acid, ammonium chloride, boceprevir, danoprevir, narlaprevir, telaprevir, or vaniprevir.

13. The method of claim 8, wherein the composition further comprises a diluent, an excipient, a carrier, or combinations thereof.

14. The method of claim 8, wherein the malady comprising arthritis is osteoarthritis.

15. The method of claim 8, wherein the placenta-derived composition comprises IL-10, SIRT1, IGF1, IGF2, BMP2, BMP7, TGF-p, or combinations thereof.

16. A method of treating an individual having irradiation damage to bone, muscle, or connective tissue with a placenta-derived composition.

17. The method of claim 16, wherein the placenta-derived composition comprises a placental tissue and one or more protease inhibitors.

18. The method of claim 17, wherein the placental tissue comprises a placental disc.

19. The method of claim 17, wherein the placental tissue is acellular.

20. The method of claim 17, wherein the one or more protease inhibitors comprises 4-(2-Aminoethyl) benzenesulfonyl fluoride hydrochloride (AEBSF), Bovine Lung Aprotinin, N-(trans-Epoxysuccinyl)-L-leucine 4-guanidinobutylamide, Leupeptin, Nethylmaleimide (NEM), phenylmethylsulfonylfluoride (PMSF), ethylenediamine tetraacetic acid (EDTA), ethylene glycol-bis-(2-aminoethyl (ether) NNN′N′-tetraacetic acid, ammonium chloride, boceprevir, danoprevir, narlaprevir, telaprevir, or vaniprevir.

21. The method of claim 16, wherein the composition further comprises a diluent, an excipient, a carrier, or combinations thereof.
